(19) United States
(12) Patent Application Publication
(10) Pub. No.: US 2025/0067732 A1
(43) Pub. Date: Feb. 27, 2025

(54) CRITICALLY-LOCKED MECHANICAL METAMATERIAL FOR HYPER-RESPONSIVE MOLECULAR PROFILING

(71) Applicant: National University of Singapore, Singapore (SG)

(72) Inventors: Haitao Zhao, Singapore (SG); Sijun Pan, Singapore (SG); Huilin Shao, Singapore (SG)

(21) Appl. No.: 18/721,809

(22) PCT Filed: Dec. 21, 2022
(86) PCT No.: PCT/SG2022/050922

§ 371 Date: Jun. 19, 2024

(30) Foreign Application Priority Data
Dec. 21, 2021 (SG) 10202114186S

(51) Int. Cl. G01N 33/543

(52) U.S. Cl. CPC G01N 33/5436; G01N 2333/4725; G01N 2333/70596; G01N 2333/908

(57) ABSTRACT

Disclosed herein is a composite material comprising:
a substrate; and
a patterned hydrogel disposed on the substrate, wherein:
   the patterned hydrogel comprises stimulus-responsive constitutional units and constitutional units comprising one or more target molecule recognition moieties;
   the stimulus-responsive constitutional units are responsive to a stimulus and are configured to produce a stress value S₁ to the patterned hydrogel when the stimulus is applied;
   the target molecule recognition moieties are responsive to a target molecule and are configured to impart a stress value S₂ to the patterned hydrogel upon interaction with the target molecule;
   the patterned hydrogel is configured to reversibly buckle and/or reversibly swell when a threshold stress level T of the patterned hydrogel is crossed.

### FIELD OF THE INVENTION

The present invention relates to a composite material, and to a method of detecting a biomolecule using the composite material.

### BACKGROUND

The listing or discussion of a prior-published document in this specification should not necessarily be taken as an acknowledgement that the document is part of the state of the art or is common general knowledge.

Mechanical metamaterials, as a recent branch of metamaterials, have received increasing attention due to their ability to dramatically alter the mechanical response of a system. These exploit carefully-structured materials to achieve exotic behaviors that cannot be attained by their material constituents alone. Organized within a rationally-designed architecture, the building blocks of these metamaterials act together with neighboring building blocks, in a collective manner, to yield unprecedented functionalities for diverse applications. Despite such promising potential, current biomedical developments remain limited and focus primarily on exploiting these metamaterials for biomechanical and/or structural support; size-matched geometries have been developed as foot grips (Babaee, S. et al., Nat. Biomed. Eng. 2020, 4, 778-786) and cell scaffolds (Wegst, U. G. et al., Nat. Mater. 2015, 14, 23-36; and Laronda, M. M. et al., Nat. Commun. 2017, 8, 15261), respectively.

Motivated by their multiple advanced behaviors, such as nonlinearities and shape-transforming capabilities, we reasoned that mechanical metamaterials can offer unique opportunities to dramatically amplify even faint biomolecular interactions. In particular, responsive hydrogels make a promising candidate to bridge biomolecular events and mechanical responses. Through materials engineering, hydrogels can be prepared in various compositions to recognize different stimuli and produce myriad mechanical responses (e.g., volume and stiffness); through advanced fabrication, hydrogels can be readily structured, patterned and integrated. Nevertheless, to develop hydrogel-based mechanical metamaterials for signal enhancement, several challenges remain. Firstly, while metamaterials can offer advanced amplification mechanisms, typically over a highly delicate range of conditions, this critical window of responsiveness is easily missed in hydrogels due to their variable cross-linking and/or patterning. Secondly, as most hydrogels rely on bulk target diffusion within the gel matrix to actuate an ensemble response, they are slow to respond and lack the ability to distinguish spatial distribution of stimuli.

Therefore, there exists a need for new mechanical metamaterials for hyper-responsive molecular profiling.

### SUMMARY OF THE INVENTION

The present invention solves some or all of the problems and needs associated with the prior art, and provides a hyper-responsive molecular profiling system that leverages post-casting tuning (to attain the critical state) and stimulus-induced geometric transformation (to enhance detection signal), which are different from the pre-casting optimization and linear volumetric change in conventional hydrogel biosensors.

Thus, the invention provides the following numbered clauses.

1. A composite material comprising:
a substrate; and
a patterned hydrogel disposed on the substrate, wherein:
   the patterned hydrogel comprises stimulus-responsive constitutional units and constitutional units comprising one or more target molecule recognition moieties;
   the stimulus-responsive constitutional units are responsive to a stimulus and are configured to produce a stress value S₁ to the patterned hydrogel when the stimulus is applied; the target molecule recognition moieties are responsive to a target molecule and are configured to impart a stress value S₂ to the patterned hydrogel upon interaction with the target molecule;
   the patterned hydrogel is configured to reversibly buckle and/or reversibly swell when a threshold stress level T of the patterned hydrogel is crossed.
   2. The composite material according to Clause 1, wherein the one or more target molecule recognition moieties comprise a moiety selected from the group consisting of a crosslinkable moiety, a cleavable crosslinking moiety, and a moiety capable of covalently bonding to a polar molecule, optionally wherein the crosslinkable moiety and/or a cleavable crosslinking moiety comprises a redox-responsive moiety.
   3. The composite material according to Clause 2, wherein the crosslinkable moiety is a moiety capable of being crosslinked by a biomolecule (e.g. an enzyme) and/or wherein the cleavable crosslinking moiety is a moiety capable of being cleaved by a biomolecule (e.g. an enzyme).
   4. The composite material according to Clause 3, wherein the biomolecule is an enzyme selected from the group consisting of an oxidase enzyme, a peroxidase enzyme, a protease and an enzyme that cleaves DNA,
optionally wherein:
   the enzyme is selected from the group consisting of thioredoxin, glutaredoxin, horseradish peroxidase (HRP), glucose oxidase, glutathione peroxidase, laccase, tyrosinase and glutathione reductase.
   5. The composite material according to any one of Clauses 2 to 4, wherein the crosslinkable moiety is capable of being crosslinked by horseradish peroxidase, optionally wherein the crosslinkable moiety comprises phenol moiety (e.g. a tyrosine moiety), a thiol moiety, a catechol moiety (e.g. a dopamine moiety or a 3,4-dihydroxybenzylamine moiety).
   6. The composite material according to any one of the preceding clauses, wherein the one or more target molecule recognition moieties comprise a moiety capable of reacting with a mixture of formaldehyde and tris(hydroxymethyl)aminomethane to form a functional group having the formula —CH₂—NHC(CH₂OH)₃,
optionally wherein the one or more target molecule recognition moieties comprise a phenol ring (e.g. a tyrosine moiety) or a catechol ring (e.g. a dopamine moiety or a 3,4-dihydroxybenzylamine moiety).
   7. The composite material according to any one of the preceding clauses, wherein the patterned hydrogel comprises constitutional units comprising a moiety (e.g. an antibody) targeted to a biomolecule selected from the group consisting of a protein biomarker, a DNA sequence, and an RNA sequence.
   8. The composite material according to Clause 7, wherein the patterned hydrogel comprises constitutional units comprising a moiety (e.g. an antibody) targeted to a biomolecule that recruits an enzyme that is capable of catalysing a crosslinking reaction and/or a cleavage reaction, optionally wherein the enzyme is selected from the group consisting of an oxidase enzyme, a peroxidase enzyme, a protease and an enzyme that cleaves DNA, more optionally wherein the enzyme is selected from the group consisting of horseradish peroxidase (HRP), glucose oxidase, glutathione peroxidase, laccase, tyrosinase and glutathione reductase.
   9. The composite material according to Clause 7, wherein the patterned hydrogel comprises constitutional units comprising a moiety (e.g. an antibody) targeted to a biomolecule selected from the group consisting of CD63, CD24, EpCAM, EGFR, MUC1, CD125, HER2 and CEA.
   10. The composite material according to any one of the preceding clauses, wherein:
the one or more target molecule recognition moieties comprise a crosslinkable moiety and/or a cleavable crosslinking moiety; and
   the patterned hydrogel comprises constitutional units comprising a bio-moiety that recruits an enzyme that is capable of catalysing a crosslinking reaction between the crosslinkable moieties, or a cleavage reaction of a cleavable crosslinking moiety, which crosslinking reaction or cleavage reaction imparts a stress value S₂ to the patterned hydrogel, optionally wherein the constitutional units comprising a crosslinkable moiety and/or a cleavable crosslinking moiety comprise a redox-responsive moiety.
   11. The composite material according to any one of the preceding clauses, wherein:
(a) the constitutional units comprising one or more target molecule recognition moieties comprise constitutional units comprising a crosslinkable moiety selected from the group consisting of a phenol moiety (e.g. a tyrosine moiety) and a thiol moiety, optionally wherein the constitutional units comprising or more target molecule recognition moieties comprise constitutional units derived from one or more of the group consisting of N-acryloyltyramine (NATA), 2-mercaptoethyl acrylate and N-(2-mercaptoethyl)acrylamide (MEAM); or
(b) the constitutional units comprising one or more target molecule recognition moieties comprise constitutional units comprising a cleavable crosslinking moiety selected from the group consisting of a disulfide moiety and a thioketal moiety, optionally wherein the cleavable crosslinking moiety is derived from N,N′-Bis(acryloyl)cystamine, N,N′-((propane-2,2-diylbis(sulfanediyl))bis(ethane-2,1-diyl))diacrylamide, or disulfanediylbis(ethane-2,1-diyl) diacrylate.
   12. The composite material according to any one of the preceding clauses, wherein the composite material comprises a stimulus-transmitting layer disposed between the substrate and the patterned hydrogel, which stimulus-transmitting layer comprises a stimulus-transmitting material capable of transmitting a stimulus to the stimulus-responsive constitutional units,
where said stimulus-responsive constitutional units are responsive to said stimulus, optionally wherein said stimulus-transmitting layer has a thickness of from 5 to 50 nm.
   13. The composite material according to Clause 12, wherein the stimulus-transmitting material is selected from one or more of the group consisting of a thermally conductive material and an electrically conductive material,
optionally wherein the stimulus-transmitting material is a thermally conductive material, more optionally wherein the thermally conductive material is a photothermally conductive material, such as a photothermally conductive material configured to apply a thermal stimulus to the stimulus-responsive constitutional units upon plasmonic heating of the photothermally conductive material.
   14. The composite material according to Clause 13, wherein the thermally conductive material is selected from one or more of the group consisting of gold, silver, copper, aluminium, CuₓS, platinum and zinc (e.g. gold),
optionally wherein the thermally conductive material is gold.
   15. The composite material according to any one of the preceding clauses, wherein the stimulus-responsive constitutional units are selected from one or more of the group consisting of thermally-responsive constitutional units, electrically-responsive constitutional units, optically-responsive constitutional units, magnetic-responsive constitutional units and pH-responsive constitutional units,
optionally wherein the wherein the stimulus-responsive constitutional units are selected from one or more of the group consisting of thermally-responsive constitutional units and pH-responsive constitutional units.
   16. The composite material according to Clause 15, wherein:
(a) the thermally-responsive constitutional units are formed from one or more of the group consisting of N-isopropylacrylamide (NIPAM), di(ethylene glycol)methylether methacrylate (DEGMA), triethylene glycol acrylate (TEGA), N-vinylcaprolactam (NVCL) and N-ethyl-N-methylacrylamide (EMA); or
(b) the pH-responsive constitutional units are formed from acrylic acid (AA), methacrylic acid (MAA), 4-vinylbenzoic acid (VBA), 2-(demethylamino)ethyl methacrylate (DMAEMA), 2-(diethylamino)ethyl methacrylate (DEAEMA), 2-vinylpyridine (2VP), 11-acrylamidoundecanoic acid (AaU) and sodium 2-acrylamido-2-methylpropanesulfonate (AMPS).
   17. The composite material according to any one of the preceding clauses, wherein the patterned hydrogel is patterned to have a lattice structure,
optionally wherein the lattice structure comprises substantially square-shaped holes.
   18. The composite material according to any one of the preceding clauses, wherein the reversible buckling and/or reversible swelling of the patterned hydrogel is detectable by scanning electron microscopy and/or laser diffraction,
optionally wherein the patterned hydrogel comprises left-handed and right-handed structures.
   19. A method of detecting a biomolecule target in a sample, comprising the steps:
(i) providing a composite material according to any one of Clauses 1 to 18, and a source of a stimulus to which the stimulus-responsive material is responsive;
(ii) applying the stimulus at a first magnitude to the composite material in the presence of said sample;
(iii) repeating step (ii) at a different stimulus magnitude to determine the threshold stress level T;
(iv) contacting the composite material with a sample and simultaneously applying the stimulus at a magnitude for which:

$${S}_{1} < T and {S}_{1} + {S}_{2} > T , or$$

$${S}_{1} > T and {S}_{1} + {S}_{2} < T ;$$

(v) determining the presence of said biomolecule target upon detecting a conformational change (e.g. change in buckling state) of the patterned hydrogel, wherein a change in buckling state indicates the presence of said biomolecule target.
   20. The method according to Clause 19, wherein the source of a stimulus to which the stimulus-responsive material is responsive is a source of thermal energy or a pH change, optionally wherein the source of thermal energy is a source of electromagnetic radiation, more optionally wherein irradiation of the stimulus-transmitting material, when present, by the source of thermal energy (e.g. a source of electromagnetic radiation) provides plasmonic heating of the stimulus-responsive material.
   21. The method according to Clause 19 or 20, wherein the buckling and/or swelling of the patterned hydrogel is determined using scanning electron microscopy (SEM).
   22. The method according to Clause 19 or 20, wherein the conformational change of the patterned hydrogel is determined using laser diffraction, optionally wherein the patterned hydrogel comprises left-handed and/or right-handed structures.
   23. The method according to any one of Clauses 19 to 22, wherein:
(a) the one or more target molecule recognition moieties comprise crosslinkable moieties; and
(b) the patterned hydrogel comprises constitutional units comprising a moiety (e.g. an antibody) targeted to a biomolecule that recruits an enzyme that is capable of catalysing a crosslinking reaction of the crosslinkable moieties,
optionally wherein the enzyme is selected from the group consisting of an oxidase enzyme, a peroxidase enzyme, a protease and an enzyme that cleaves DNA,
more optionally wherein the enzyme is selected from the group consisting of horseradish peroxidase (HRP), glucose oxidase, and glutathione peroxidase.
   24. The method according to Clause 23, wherein the patterned hydrogel comprises constitutional units comprising a moiety (e.g. an antibody) targeted to a biomolecule selected from the group consisting of CD63, CD24, EpCAM, EGFR, MUC1, CD125.
   25. The method according to Clause 23 or 24, wherein the crosslinkable moieties comprise tyrosine moieties, and where the enzyme is horseradish peroxidase (HRP).

### BRIEF DESCRIPTION OF THE FIGURES

FIG. 1 depicts the synthesis and characterization of redox-responsive monomer. (a) Schematics on the syntheses of N-acryloylphenethylamine (NAPEA), N-acryloyltyramine (NATA) and N-acryloyldopamine (NADA). These monomers were prepared through acryloylation of phenethylamine, tyramine and dopamine, respectively. (b)¹H NMR characterization, confirming the identity and purity of these monomers.

FIG. 2 depicts the fabrication of the hydrogel metamaterial in a microfluidic system. (a) Cross-sectional illustration of the fabrication flow. The process includes: (1-4) PDMS mould fabrication, (5-6) hydrogel patterning on Au-coated glass, and (7) bonding with microfluidic chip. (b-c) Scanning electron microscopy (SEM) images of the patterned hydrogel metamaterials in the breathing state (b) and buckling state (c). Insets show the magnified view of the hydrogel metamaterials. (d) Statistical analysis of the metamaterial hole morphology. With increasing swelling, the metamaterial demonstrated geometric changes. In the breathing state, square-hole morphology was preserved; the holes showed an average side length of 11.24±0.13 μm (top). In the buckling state, mutually orthogonal rectangular holes were formed, with an average length of 16.66±0.28 μm and an average width of 8.54±0.17 μm (bottom).

FIG. 3 depicts the critically-locked mechanical metamaterial for amplified molecular profiling. (a) Schematic of the mechanical metamaterial operating at critical point for hyper-responsive analysis (MORPH) platform. The technology is designed to enhance the hydrogel's responsiveness to biomolecular stimuli. The scheme includes: (1) metamaterial patterning. Dual-responsive hydrogel, which comprises N-isopropylacrylamide (NIPAM) as the temperature-responsive monomer, N-acryloyltyramine (NATA) as the redox-responsive monomer and antibody monomer for molecular recognition, was patterned into square-hole lattices on a gold-coated glass (Au—SiO₂) substrate; (2) critical-point locking. The patterned metamaterial was then precisely locked to its critical transition state through LED-activated plasmonic heating at the gold-hydrogel interface; and (3) chiral transformation. When target biomarkers are introduced, free radicals are generated through antibody-peroxidase activity to induce further hydrogel cross-linking. This mechanical perturbation rapidly breaks the transition state and triggers a dramatic chiral transformation of the metamaterial, leading to amplified changes in the projected optical diffraction. (b) Metamaterial deformations and optical signals. When the metamaterial is unlocked, biomarker-induced swelling only induces minimal deformations and thus the optical diffraction signal is small. However, when the metamaterial is critically-locked (near its critical point, CP), an equal amount of biomarker-induced swelling triggers the rapid release of the accumulated strain energy and induces a dramatic chiral pattern transformation. This geometric reorganization causes a distinct mode change in the projected diffraction pattern, thereby enabling amplified optical detection of even scarce biomolecules. (c-e) Photographs of the microfluidic device and the smartphone-based optical detector. The miniaturized microfluidic system and optical detector were designed to streamline the MORPH assay workflow and facilitate optical diffraction measurements. Scale bar: 1 cm.

FIG. 4 depicts the NATA monomer selection. The synthesized monomers (a) NAPEA, (b) NATA and (c) NADA were incorporated at the same concentration to form respective hydrogels, and were evaluated for their ability to induce hydrogel cross-linking, when being treated with horseradish peroxidase (HRP) and hydrogen peroxide (H₂O₂) which generate free radicals. Due to their varying number of hydroxyl groups, the monomers showed different response kinetics. The NAPEA-incorporated hydrogel showed little response to HRP, while the NADA-incorporated hydrogel was labile and showed hydrogel shrinking spontaneously in water, even in the absence of HRP. In comparison, NATA showed a good stability in water (i.e. negligible spontaneous swelling changes in the absence of HRP) and a large response to HRP-generated free radicals. NATA was thus selected as the redox-responsive monomer for metamaterial formation. All measurements were performed in triplicate and the data are presented as mean±s.d.

FIG. 5 depicts the molecular and geometric changes in the metamaterial during the MORPH workflow. (a) The hydrogel precursors are mixed and patterned under UV light. The cured metamaterial is formed in its breathing state, through alkene-based polymerization, and structured as a periodic array of square-holes. Different stimulus-responsive functional groups are not reacted. (b) During plasmonic tuning, plasmon-phonon coupling generates heat to change the hydrogen bonding in the hydrogel metamaterial (through the NIPAM functional group). By controlling the amount of plasmonic heating, the metamaterial is thus being tuned and locked to its transition state, where it experiences substantial molecular-level mechanical perturbation yet preserves its geometric morphology. (c) In the presence of biomarkers, horseradish peroxidase (HRP) generates free radicals to cause further cross-linking in the hydrogel metamaterial (through the NATA functional group). This biomarker-induced mechanical perturbation rapidly triggers a cooperative buckling in the metamaterial, leading to a dramatic pattern transformation of the metamaterial geometry.

FIG. 6 depicts the exploded view of the microfluidic device. The device 100 was assembled from a cover layer 610 (poly(methyl methacrylate) (PMMA)), a microchannel layer 620 (double-sided tape) with preloaded reagents 602, and an Au-coated substrate layer 630 (glass) with patterned hydrogel metamaterial 605 on the surface.

FIG. 7 depicts the smartphone-based optical detector. (a) Mechanical design of the optical detector. It consists of four building blocks: (1) the holder for LED and driving circuits; (2) the holder for laser diode, beam splitter and battery; (3) sliding slot for chip loading and the holder for magnification lens; and (4) casing and smartphone holder. The four blocks are assembled through click-fit mechanisms. (b) Photographs of the individual blocks before assembly: (1) LED and driving circuits; (2) laser diode and beam splitter; (3) magnification lens; and (4) casing and smartphone holder. (c) Correlation of MORPH measurements by the smartphone-based detector and commercially available plate reader. The smartphone-based detector correlated well with the commercial reader (R²=0.983). All measurements were performed in triplicate and the data are presented as mean±s.d.

FIG. 8 depicts the critical point in pattern transformation. (a) Hydrogel metamaterial in the breathing, transition, and buckling state. With increasing swelling, the metamaterial's square-hole morphology was preserved in the breathing and transition state, before rapidly collapsing into mutually orthogonal rectangular holes in the buckling state. Scale bar: 10 μm. (b) Metamaterial deformation as a function of swelling ratio (SR). The deformation index (DI) increased minimally and linearly at a small SR; whereas at a threshold SR (critical point, CP), it showed an abrupt increase upon further swelling. A similar trend was observed across metamaterials with various hydrogel compositions. (c) Metamaterial deformation change as a function of initial SR. With a fixed 5% increase in SR, the deformation index change (ΔDI) was maximal when the initial SR was precisely matched to the metamaterial's critical point. (d) Theoretical modeling of critical points with respect to various pre-casting factors. We observed that the critical point increases with increasing shear modulus and periodicity of the metamaterial. (e) Experimental tuning of critical points by adjusting the pre-casting factors. We varied the metamaterial's shear modulus by increasing the doping of NATA monomer and its pattern periodicity by changing the casting mold. While the experimental tuning showed similar trends to that of theoretical modeling, it showed considerable variations even among technical replicates. The large variability highlights the experimental challenges in critical-tuning the metamaterials solely through these pre-casting factors. All data are presented as mean±s.d. The measurements in (b) and (c) were repeated three times, and the measurements in (e) were repeated five times.

FIG. 9 depicts the nonlinear finite element simulation of a pattern transformation. (a) Simulated deformation patterns of a mechanical metamaterial, under a range of swelling strains. When the swelling is small (strain=0), the metamaterial swells linearly and the square-hole morphology is preserved (breathing state). When the swelling is increased to the critical condition (strain=0.076), subtle disturbance starts to appear in the metamaterial but the square hole morphology is still maintained (transition state). When the swelling is further increased (strain=0.087 and 0.139), the metamaterial starts to buckle and the square holes collapse into mutually orthogonal rectangular holes (buckling state). (b) Simulation of the displacement at P₀ as a function of strain. The rate of metamaterial deformation increases sharply near the transition state. (c) Simulation of Mises stress at P₁ and P₂ as a function of strain. The accumulated stress is suddenly released and converted into mechanical deformation during the pattern transformation.

FIG. 10 depicts the SR and DI during a pattern transformation. (a) Schematic illustration of a deformation pattern. SR is defined as the normalized volumetric change in the hydrogel metamaterial. DI is defined as the average length/width change, and is used to characterize geometric changes of the hydrogel metamaterial during a pattern transformation. (b-f) Photographs of the hydrogel metamaterial during a pattern transformation. All indicated SRs and DIs were measured experimentally.

FIG. 11 depicts the plasmonic locking of critical point. (a) Illustration of the plasmonic modulation. Through LED-activated plasmonic heating at the gold-hydrogel interface, the temperature-responsive hydrogel metamaterial is rapidly and precisely heated, thereby inducing a distinct swelling change to tune the metamaterial to its desired swelling state. (b) Measured optical absorption spectra for a range of gold-film thickness. The 10 nm gold film was chosen for good overall transmission; the absorption peak at λ˜440 nm was used for enhanced absorption in plasmonic locking, and the absorption valley at λ˜520 nm was used to boost transmission in MORPH interferometric measurements. (c) Simulated kinetics of plasmonic heating. The heat generated at the gold layer could be rapidly transferred throughout the hydrogel. (d) Deformation response to “on” and “off” LED illuminations. The metamaterial showed a rapid, repeatable and precise response to plasmonic modulation. (e) Versatile plasmonic modulation to amplify different types of stimulus-induced deformation changes. To amplify swelling changes, we tuned and locked the metamaterial from its initial state (P_{before}) to its critical point (P_{lock}) by applying a large LED current (I_{LED}, mA). The stimulus-induced swelling drove the metamaterial to its final state (P_{after}) and achieved a large deformation increase. To amplify cross-linking changes, we critically tuned the metamaterial to the locked state (P_{lock}) with a reduced LED current. The stimulus-induced cross-linking resulted in a highly contracted final state (P_{after}, also the critical point, CP) and thus demonstrated a large deformation decrease. The graphs show schematic guides to illustrate typical relationships between DI and SR. (f) Comparison of the metamaterial response with and without plasmonic locking. The critically-locked platforms demonstrated broad applicability to amplify various stimulus-induced deformation changes, regardless of the initial swelling states (P_{before}) of the systems. All measurements were performed in triplicate and the data are presented as mean±s.d in (e) and (f).

FIG. 12 depicts the characterization of LED-activated plasmonic modulation. (a) Swelling properties of hydrogels made with different NIPAM concentration. The hydrogel composition with 50% w/w NIPAM was selected to enable a larger range of thermal modulation. (b) Modulation of metamaterial deformation by tuning the LED current. To control the plasmonic modulation, the LED current was increased from 50 mA to 250 mA, at a step of 20 mA, and then decreased stepwise to 50 mA. The resultant metamaterial deformation (DI) showed a rapid and repeatable response to the applied LED current change. (c) Precision of the plasmonic modulation. A good correlation (R²=0.9802) was observed between the measured deformation, as tuned through plasmonic modulation, and the target deformation. All measurements were performed in triplicate and the data are presented as mean±s.d.

FIG. 13 depicts the sequential plasmonic modulation. Sequential plasmonic modulation was achieved through modulating the LED current. The metamaterial deformation reached a plateau in 16.3 s when the LED source was turned on and returned to the initial state in 14.4 s when the LED source was turned off.

FIG. 14 depicts the kinetics of hydrogel swelling at the microscale and macroscale. (a) Photographs of a hydrogel micro-pillar before and after (60 s) immersing in water. (b) Swelling kinetics of two hydrogel compositions at the microscale. The hydrogel with the NATA monomer (square markers) demonstrated a smaller swelling change than that without the NATA monomer (circle markers). Both hydrogels reached an equilibrium in ˜60 s. (c) Photographs of a hydrogel droplet before and after (24 h) immersing in water. (d) Swelling kinetics at the macroscale. The two hydrogels reached an equilibrium in ˜10 h. The results suggest that hydrogel swelling at the microscale is significantly faster than that at the macroscale. All measurements were performed in triplicate and the data are presented as mean±s.d. in (b) and (d).

FIG. 15 depicts the metamaterial response without plasmonic modulation. (a) Mechanisms of chemical-induced swelling and cross-linking changes. To induce swelling changes, formaldehyde (CH₂O) and tromethamine (Tris) were added to introduce hydrophilic hydroxy group on NATA residues. To induce cross-linking changes, horseradish peroxide (HRP) was added to generate free radicals to enable cross-linking of NATA residues. (b) Metamaterial deformation changes without plasmonic locking. Without plasmonic locking (LED is off), all systems were used directly (P_{before}). In response to the applied chemical stimuli, the unlocked systems showed small changes in their deformation index (ΔDI), ΔDI=0.025 (swelling) and 0.038 (cross-linking); both changes were smaller than that by the critically-locked systems (FIG. 11e). Star indicates the critical point (CP). All measurements were performed in triplicate and the data are presented as mean±s.d.

FIG. 16 depicts the MORPH performance with different storage conditions. The metamaterial deformation at the initial state (P_{before}), locked state (P_{lock}), and final state (P_{after}) after different storage conditions: (a) relative humidity (RH); (b) pH; and (c) storage time at 4° C. To investigate the influence of humidity, MORPH devices were stored in a dry box (RH=38.2%), at room condition (RH=55.1%), and water bath (RH=97.8%) for 24 h. To investigate the influence of solution pH, devices were respectively stored in PBS solutions with pH=6.4, 7.4, and 8.4, for 24 h. To investigate the influence of storage time, devices were stored at 4° C. for 1 day, 1 week and 2 weeks, respectively. After storage, all devices were critically-tuned and measured at standard laboratory conditions. The results showed that upon different storage, the devices showed variable initial states (P_{before}). After plasmonic modulation, the devices were tuned to their critical state and showed a comparable signal (P_{lock}). Subsequent stimulus application caused the devices to reach a comparable final state (P_{after}) and thus showed a similar response (ΔDI). All measurements were performed in triplicate and the data are presented as mean±s.d.

FIG. 17 depicts the interferometric projection of metamaterial deformation. (a) Illustration of chiral interferometric measurement using the critically-locked metamaterial. The hydrogel metamaterial is exploited as a diffraction mask that comprises a 2D array of handed cross-structures. Laser beams passing through the two opposite-handed structures interfere with each other to create a diffraction pattern. (b-c) Simulation and experiment results of the resultant diffraction patterns in the breathing state (b) and buckling state (c). In the breathing state, the hydrogel metamaterial functions as two orthogonally-placed diffraction gratings, resulting in a classic 2D diffraction pattern. In the buckling state, adjacent left-handed and right-handed structures rotate (e) in opposite directions to form a chiral interferometer, resulting in a diffraction pattern with new hotspots (circled). Dashed line indicates the position of intensity acquisition for subsequent MORPH analysis, where H₀ and H₁ indicate the control and new hotspots, respectively. Scale bar (left): 10 μm. Scale bar (right): 200 μm. (d) Experimental intensity profiles measured along the dashed line, confirming the emergence of new optical hotspots. (e) Relationship between the optical intensities at H₁ and metamaterial deformations. As validated by both simulation and experimental results, the intensities of these emerging hotspots are strongly correlated to the metamaterial deformations (R²=0.987). (f) Resultant optical intensity changes at different metamaterial locking states. Using plasmonic modulation, we prepared the metamaterial mask in different locking states (P_{lock}), before subjecting it to an applied stimulus (HRP which generates free radicals to induce hydrogel cross-linking). The system established a maximal optical intensity change when the metamaterial deformation index was locked to 0.13. (g) Real-time optical sensorgrams of the critically-locked and unlocked systems. The critically-locked metamaterial showed rapid and amplified optical signal, upon being treated with the HRP stimulus. All measurements were performed in triplicate and the data are presented as mean±s.d in (e) and (f). a.u., arbitrary unit.

FIG. 18 depicts the relationship between metamaterial deformation and chiral rotation. Illustration of the metamaterial in the (a) breathing state and (b) buckling state. (c) Theoretical relationship between the metamaterial deformation index and its chiral rotation angle.

FIG. 19 depicts the simulated diffraction patterns of the metamaterial at different chiral rotation angles. The chiral rotation angle (θ) was increased from 0 to 45°. The light intensity at H₁ showed a strong correlation with the chiral rotation angle.

FIG. 20 depicts the experimental correlation of the MORPH optical signal with metamaterial deformation. (a-f) During a pattern transformation, the metamaterial deformation (top panel) was characterized through both its DI as well as the corresponding chiral rotation angle (θ). During the transformation, the measured optical diffraction pattern (bottom panel) showed new hotspots (circled). The intensities of these hotspots increased with increasing metamaterial deformation (θ<20.7°; DI<0.22) and then decreased gradually when the metamaterial deformation was further increased (θ>20.7°; DI>0.22).

FIG. 21 depicts the influence of the laser source on plasmonic locking. (a) Optical diffraction signal at different laser intensities. Under a constant LED illumination for plasmonic locking, the optical diffraction signal remained constant with a low laser intensity (s 0.29 mW/cm²) but decreased under a high laser intensity, possibly due to considerable laser-induced heating. (b) Relationship between the optical diffraction signal and the laser intensity. The result confirms that at the intensity of the miniaturized laser diodes, as used in the MORPH assay setup, the laser source showed negligible influence on the metamaterial deformation and thus the optical signal. The optical signal was normalized with the laser output power.

FIG. 22 depicts the biomarker distribution in complex vesicle mixtures. (a) Vesicles derived from single cell lines were characterized through enzyme-linked immunosorbent assay (ELISA) measurements. These vesicle solutions showed a similar CD63 abundance but different EpCAM expression (i.e. MKN45, high; PC9, medium; and GLI36, low). (b) Complex vesicle mixtures were prepared by combining high-expression MKN45 exosome solution with low expression GLI36 exosome solution (Mixture 1, top panel) and medium-expression PC9 exosome solution with low expression GLI36 exosome solution (Mixture 2, bottom panel). Both mixtures were adjusted to match in vesicle counts. Both mixtures showed indistinguishable ELISA profiles, indicating that they contained similar total biomarker abundance despite differences in biomarker distribution. (c) Real-time MORPH measurements (EpCAM) on these mixtures, after serial sample dilutions. (d) MORPH amplitude analysis correlates with vesicle counts (total biomarker abundance). (e) MORPH slope analysis shows correlation with biomarker distribution in the mixtures. All measurements were performed in triplicate, normalized against respective sample-matched IgG isotype control antibodies, and the data are displayed as mean±s.d.

FIG. 23 depicts the operation of the MORPH platform.

FIG. 24 depicts the multimodal characterization of extracellular vesicles. (a) Unimodal size distribution of vesicles derived from HCT116 cell line, as determined by nanoparticle tracking analysis (NTA). Inset shows the transmission electron micrograph of a vesicle. (b) Western blotting analysis of the vesicle lysate. The lysate was immunoblotted for exosomal markers (CD63, LAMP-1, Alix, HSP90, HSP70, Flotillin 1, and TSG101).

FIG. 25 depicts the MORPH assay configuration. MORPH uses two antibodies (capture and detection) to form a sandwich configuration to detect the co-localization of two biomarkers on the same vesicles.

FIG. 26 depicts the MORPH for clinical exosome profiling. (a) Schematic of MORPH profiling of biomarker abundance and distribution in exosomes. Exosomes are first immuno-captured onto the critically-locked metamaterial through anti-CD63 antibodies, before being incubated with HRP-conjugated detection antibodies. For comparison of overall biomarker abundance (top), a vesicle mixture that expresses a higher biomarker amount recruits more peroxidase enzymes to generate free radicals, thereby inducing a larger deformation in the critically-locked metamaterial. For comparison of biomarker distribution (bottom), in a vesicle mixture that expresses more densely-localized biomarkers, more peroxidase enzymes are locally recruited to generate a high local concentration of free radicals, thereby inducing faster metamaterial deformation. (b) MORPH response profiles. As compared with the unlocked platform, the critically-locked metamaterial not only demonstrated an enhanced optical signal (amplitude) that correlates with total biomarker abundance, but also showed a rapid reaction kinetics (slope) that distinguishes different biomarker distribution. Dotted lines indicate the positions of amplitude and slope measurements. (c) Detection sensitivity. The limit of detection (LOD) was determined by titrating a known amount of exosomes and analyzing the CD63 signal. 10 μL and 100 μL of samples were used in MORPH (locked and unlocked) and ELISA measurement, respectively. The critically-locked MORPH platform showed an improved performance than conventional ELISA and the unlocked assay. The dashed line shows the LOD, defined as 3×s.d. of MORPH signal in a no-sample control. Signals above the LOD are considered distinguishable with >99% confidence. (d) Correlation of MORPH and ELISA measurements. Using exosomes derived from different cell origins, we measured the expression levels of exosome marker CD63 and putative cancer markers (CD24, EpCAM, and MUC1). The MORPH analysis showed a good correlation with conventional ELISA (R²=0.947). (e) MORPH analysis of clinical ascites samples. Ascites samples from cancer patients (n=38) were measured directly with the MORPH platform, for their respective biomarker amplitude and slope analyses. (f-g) Statistical analysis of the clinical measurements. The MORPH's combined signature (i.e. biomarker amplitude and slope) showed the best accuracy in prognosis classification. All measurements were performed in triplicate and the data are presented as mean±s.d in (c-d) and as mean in (e-f). (*P<0.05, **P<0.005, ****P<0.00005, Student's t-test). a.u., arbitrary unit.

FIG. 27 depicts the comparison between MORPH and ELISA analyses. (a) ELISA kinetic profiles. ELISA analysis showed indistinguishable kinetic profiles for vesicle mixtures that bear different biomarker distribution states. (b) Comparison of assay mechanism of MORPH and ELISA. In the presence of biomarkers, MORPH generates short-lived and localized free radicals and measures the radical-enhanced hydrogel deformation; ELISA generates long-lived and diffusive luminol products and measures their chemiluminescence emission. As such, MORPH has a higher temporal and spatial resolution than ELISA. (c) MORPH and (d) ELISA analysis of the abundance of CD63, CD24, EpCAM and MUC1 on vesicles derived from different human cancer cell lines. All measurements were performed in triplicate, normalized against respective sample-matched IgG isotype control antibodies, and the data are displayed as mean.

FIG. 28 depicts the MORPH kinetic response profiles. Using the method as detailed in FIG. 22, we prepared three groups of vesicle mixtures with (a) high, (b) medium, and (c) low biomarker abundance, and in each group, samples bearing different biomarker distribution states. Across all groups, MORPH kinetic profiles could distinguish between different biomarker distribution states (localized vs. distributed).

FIG. 29 depicts the comparison of MORPH and surface plasmon resonance (SPR) sensors.

FIG. 30 depicts the specificity analysis of the MORPH platform. (a) MORPH performance with different chemical agents and physical effects. We prepared exosomes in buffers with different biological background (PBS vs. vesicle-depleted ascites), varying ionic strength (0.1×PBS vs. 1×PBS) and pH (pH=6.4 vs. pH=7.4), and performed the experiments at different temperatures (20° C. vs. 25° C.). (b, c) MORPH response profiles with exosomes spiked in (b) PBS buffer and (c) vesicle-depleted ascites. For all conditions tested, we used anti-CD63 capture (positive) and IgG isotope control antibody (negative) to measure the prepared samples. The positive measurements showed a consistent and higher signal while the negative measurements showed a comparable and lower signal, demonstrating the high specificity of the system. All measurements were performed in triplicate and the data are presented as mean±s.d. in a. a.u., arbitrary unit.

FIG. 31 depicts the MORPH activation by low vesicle counts. (a) MORPH was treated with a low amount of vesicles (˜2×10⁴), spiked in PBS and vesicle-depleted ascites, respectively. In both samples, MORPH showed efficient and repeatable activation, with a low coefficient of variation (Coefficient of Variation (CV)=4.32% in PBS and 6.11% in ascites). (b) Amplitude and slope analysis of the MORPH response. MORPH demonstrated comparable activation in both PBS and vesicle-depleted ascites. Positive measurements were performed with anti-CD63 capture and sample-matched controls with IgG isotope control antibodies. All measurements were performed in triplicate and the data are presented as mean±s.d in b. (**P<0.005, ***P<0.0005, n.s., not significant, Student's t-test). a.u., arbitrary unit.

FIG. 32 depicts the clinical sample analysis. (a) Vesicle counts of clinical ascites, as determined by NTA. Receiver operating characteristic curve based on (b) CD63 expression and vesicle concentration, (c) ELISA measurement, (d) the amplitude measurement and (e) the slope measurement of individual biomarkers (CD63, CD24, EpCAM and MUC1). The combined MORPH signature (area under curve (AUC)=0.941) outperformed vesicle concentration-based analysis (AUC=0.546), ELISA analysis (AUC<0.689), and that of individual biomarkers (i.e. amplitude or slope) (AUC<0.773). All measurements were performed in triplicate, and the data are displayed as mean±s.d. in (a).

FIG. 33 depicts the MORPH classification of clinical prognosis. (a) MORPH signature scores for all patient samples. The dashed line indicates the Youden's index for defining the optimal threshold. (b) The MORPH signature showed 100% sensitivity (21/21), 88.2% specificity (15/17) and an accuracy of 94.7% (36/38) in differentiating cancer patient prognosis. All measurements were performed in triplicate and the data are presented as mean±s.d in a. a.u., arbitrary unit.

FIG. 34 depicts that the metamaterial made of pH responsive hydrogel was in the breathing state at low pH (pH=2), and gradually changed into the buckling state when the solution pH was increased (pH=3 & 4).

### DETAILED DESCRIPTION OF THE INVENTION

The invention provides a composite material comprising:
a substrate; and
a patterned hydrogel disposed on the substrate, wherein:
   the patterned hydrogel comprises stimulus-responsive constitutional units and constitutional units comprising one or more target molecule recognition moieties;
   the stimulus-responsive constitutional units are responsive to a stimulus and are configured to produce a stress value S, to the patterned hydrogel when the stimulus is applied;
   the target molecule recognition moieties are responsive to a target molecule and are configured to impart a stress value S₂ to the patterned hydrogel upon interaction with the target molecule;
   the patterned hydrogel is configured to reversibly buckle and/or reversibly swell when a threshold stress level T of the patterned hydrogel is crossed.

The word “comprising” may be interpreted herein as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The phrase, “consists essentially of” and its pseudonyms may be interpreted herein to refer to a material where minor impurities may be present. For example, the material may be greater than or equal to 90% pure, such as greater than 95% pure, such as greater than 97% pure, such as greater than 99% pure, such as greater than 99.9% pure, such as greater than 99.99% pure, such as greater than 99.999% pure, such as 100% pure.

As used herein, the singular forms “a,” “an,” and “the” include plural referents unless the context clearly dictates otherwise. Thus, for example, reference to “a composition” includes mixtures of two or more such compositions, reference to “an oxygen carrier” includes mixtures of two or more such oxygen carriers, reference to “the catalyst” includes mixtures of two or more such catalysts, and the like.

The substrate may be any suitable substrate, for example any suitable solid transparent material. Specific examples of suitable substrates include glass quartz and sapphire.

As used herein, a “hydrogel” refers to a material comprising three-dimensional crosslinked polymer network that is able to swell in the presence of water. The hydrogels useful in the invention comprise stimulus-responsive constitutional units and constitutional units comprising one or more target molecule recognition moieties. As will be understood by a person skilled in the art, the patterned hydrogels useful in the invention are crosslinked, and this crosslinking may be between the stimulus-responsive constitutional units only, the constitutional units comprising one or more target molecule recognition moieties only, other constitutional units present in the hydrogel (e.g. to provide crosslinks), or any suitable combination thereof. For example, the hydrogels useful in the invention may also comprise further constitutional units, such as crosslinkable constitutional units that may comprise redox-responsive moieties.

The composite material of the invention comprises a patterned hydrogel. As used herein a “patterned hydrogel” is a hydrogel that has a specific shape or three-dimensional bulk structure that may be distorted by buckling or swelling. For example, the patterned hydrogel may have a lattice shape/structure, where buckling and/or swelling of the patterned hydrogel causes distortion of the lattice. This distortion may be detectable using a microscope, for example scanning electron microscopy, and/or by laser diffraction through the lattice. The patterned hydrogel may comprise left handed and right handed structures that will cause differences in laser diffraction when plane-polarised light is used. This may enable the detection of buckling and/or swelling at specific locations in the patterned hydrogel.

As explained in more detail herein:
the patterned hydrogel comprises stimulus-responsive constitutional units and constitutional units comprising one or more target molecule recognition moieties;
the stimulus-responsive constitutional units are responsive to a stimulus and are configured to produce a stress value S, to the patterned hydrogel when the stimulus is applied;
the target molecule recognition moiety is responsive to a target molecule and is configured to imparts a stress value S₂ to the patterned hydrogel upon interaction with the target molecule;
the patterned hydrogel is configured to reversibly buckle and/or reversibly swell when a threshold stress level T of the patterned hydrogel is crossed.

In other words, the hydrogel has a native state in which it is not buckled or swollen. The hydrogel will buckle or swell (or unbuckle or unswell) if a stress level of the hydrogel crosses a critical value, T.

Two sources of stress are explicitly envisaged herein.
The first is a stress resulting from stimulating the stimulus-responsive constitutional units of the hydrogel (for example by heating thermally responsive constitutional units by applying light to light responsive constitutional units, or by changing the environment pH for pH-responsive constitutional units). This imparts a stress of value S, to the patterned hydrogel. The magnitude of the stress value S is typically controllable by controlling the magnitude of the stimulus, for example by controlling the temperature, light intensity or pH. In this way, a number of stress values S may be imparted into the patterned hydrogel, which enables the determination of the threshold stress level, T.
A second source of stress is a stress resulting from interaction of the target molecule recognition moieties with their target molecules, which results in a stress value S₂. This stress may be caused, for example, by interaction with a target molecule resulting in crosslinking of crosslinkable groups within the hydrogel, or resulting in a change in swelling state of the hydrogel.
   The crosslinking of crosslinkable groups may be caused by interaction between crosslinkable groups in the hydrogel with a target molecule that is able to crosslink the crosslinkable groups, which may be the case when the target molecule is an enzyme (e.g. horseradish peroxidase). In some cases, the hydrogel may comprise moieties that are targeted to biomolecules that recruit such an enzyme (e.g. the hydrogel may comprise an antibody (such as anti-CD63), which antibody targets a biomolecule (e.g. targets a protein on the surface of an exosome), where the biomolecule directly or indirectly recruits such an enzyme. In this context, indirectly recruits is intended to cover a scenario where the antibody targets a biomolecule on the surface of another biomolecule, such as an exosome, where the exosome also comprises a protein or other biomolecule that recruits the enzyme.). Recruitment of an enzyme that crosslinks the hydrogel may allow for benefits such as more localised crosslinking of the hydrogel and higher detection sensitivity.
   A change in the swelling state of the hydrogel may be caused by a change in the polarity of surface functional groups in the hydrogel that result a change in hydrogel bonding/solvation of surface functional groups. For example, swelling may occur when the hydrogel becomes more hydrophilic. Such a change may be caused by a reaction of functional groups in the hydrogel, for example reaction with a mixture of formaldehyde and tris(hydroxymethyl)aminomethane to form a functional group having the formula —CH₂—NHC(CH₂OH)₃, which functional group comprises three hydroxy moieties that are able to form strong interactions with water.
   A change in the swelling state of the hydrogel may also be caused by cleavage of crosslinking groups in the hydrogel, which may result in effects that are essentially the opposite of those discussed above in relation to crosslinking. As explained above in relation to crosslinking, this cleavage may be an enzymatic cleavage and may take place in an analogous manner to enzymatic crosslinking.

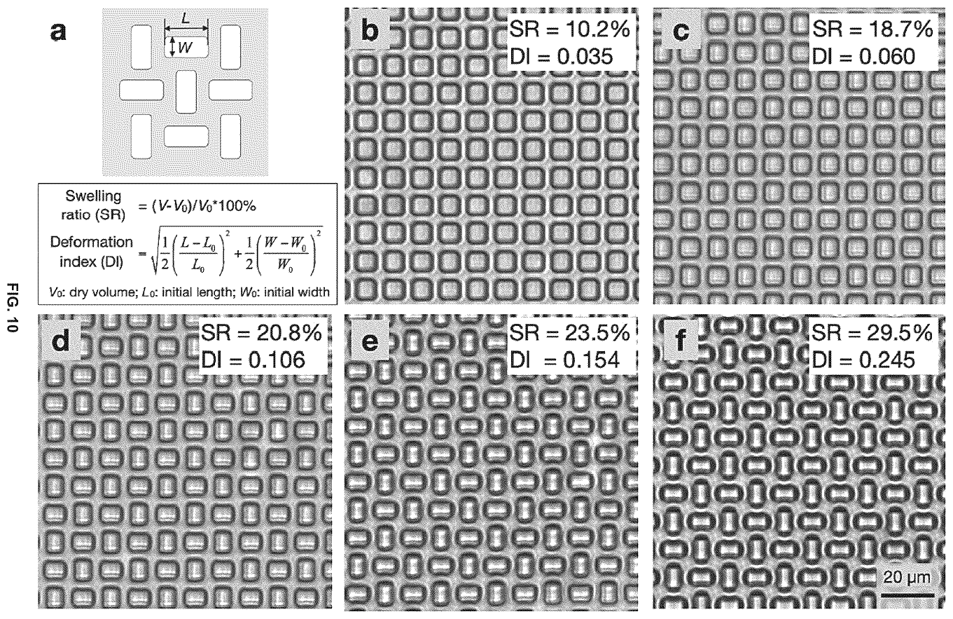

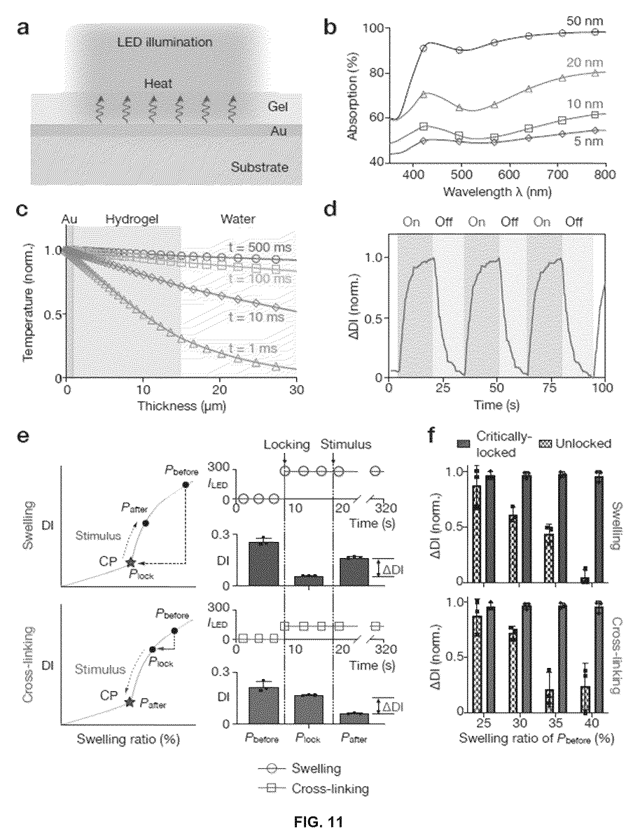

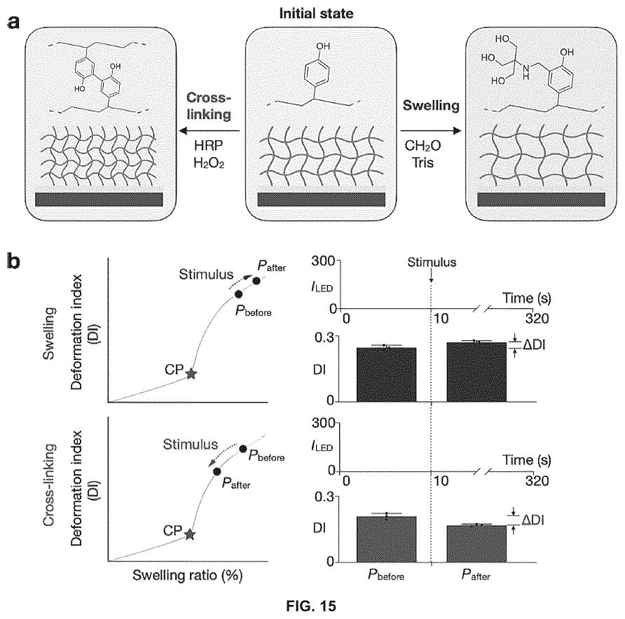

These changes are shown in FIGS. 11e and 15.

The magnitude of the stress value S₂ is typically small, such that application of S₂ alone will not usually cause the threshold stress level, T, to be crossed. However, if the patterned hydrogel is stressed by application of a stimulus causing a stress value S, that is close to the threshold stress level, T (whether above or below the threshold), then the threshold may be crossed upon application of stress value S₂, which may be positive or negative depending on the nature of the stress.

In some embodiments of the invention that may be mentioned herein, the one or more target molecule recognition moieties may comprise a moiety selected from the group consisting of a crosslinkable moiety, a cleavable crosslinking moiety, and a moiety capable of covalently bonding to a polar molecule. In some embodiments of the invention, the crosslinkable moiety and/or a cleavable crosslinking moiety may comprise a redox-responsive moiety, such as a phenol moiety (e.g. a tyrosine moiety), a thiol moiety, a catechol moiety (e.g. a dopamine moiety or a 3,4-dihydroxybenzylamine moiety). Particular examples of redox-responsive moieties include a phenol moiety (e.g. a tyrosine moiety), and a thiol moiety.

The crosslinkable moiety and cleavable crosslinking moiety may be crosslinkable or cleavable by a biomolecule, such as an enzyme. Examples of enzymes that may be useful in crosslinking, or cleavage, reactions include an oxidase enzyme, a peroxidase enzyme, a protease and an enzyme that cleaves DNA. Specific examples of enzymes include thioredoxin, glutaredoxin, horseradish peroxidase (HRP), glucose oxidase, glutathione peroxidase, laccase, tyrosinase and glutathione reductase. Particular examples of enzymes include thioredoxin, glutaredoxin, horseradish peroxidase (HRP), glucose oxidase, and glutathione peroxidase. A person skilled in the art will understand which of these enzymes will be suitable for which types of reactions disclosed herein.

As disclosed herein, the composite material may comprise constitutional units that comprise crosslinkable moieties, such as moieties that are capable of being crosslinked by enzymes, e.g. horseradish peroxidase. As used herein, “capable of being crosslinked by an enzyme” refers to a moiety being capable of being crosslinked by an enzyme under the conditions in which such an enzyme is able to perform crosslinking reactions. By way of example, in the case of horseradish peroxidase, such conditions typically involve the presence of hydrogen peroxide. A skilled person would understand the relevant conditions for other enzymes that are able to catalyse crosslinking reactions.

Thus, in some embodiments of the invention that may be mentioned herein, the crosslinkable moiety may be capable of being crosslinked by a peroxidase, such as horseradish peroxidase.

A skilled person will be aware of various functional groups that may be crosslinked by horseradish peroxidase (and other peroxidases), as well as by other enzymes.

Therefore, in some embodiments of the invention that may be mentioned herein, the crosslinkable moiety may be selected from the group consisting of a phenol moiety (e.g. a tyrosine moiety), a thiol moiety, a catechol moiety (e.g. a dopamine moiety or a 3,4-dihydroxybenzylamine moiety). Particular examples of crosslinkable moieties include a phenol moiety (e.g. a tyrosine moiety), and a thiol moiety.

As disclosed herein, the composite material may comprise constitutional units that comprise a cleavable crosslinking moiety. It is to be understood that a reference to a cleavable crosslinking moiety in this context refers to a cleavable crosslinking moiety that is covalently bonded to said constitutional unit at one end, where the other end of the cleavable crosslinking moiety is covalently bonded to a different constitutional unit. In other words, where a cleavable crosslinking moiety crosslinks a first constitutional unit with a second constitutional unit, both the first and second constitutional units may be described as comprising a cleavable crosslinking moiety.

Examples of cleavable crosslinking moieties that may be mentioned herein include a cleavable crosslinking moiety selected from the group consisting of a disulfide moiety and a thioketal moiety. Therefore, in some embodiments of the invention that may be mentioned herein, the cleavable crosslinking moiety may be selected from the group consisting of a disulfide moiety and a thioketal moiety. In some embodiments of the invention that may be mentioned herein, the cleavable crosslinking moiety may be derived from N,N′-Bis(acryloyl)cystamine, N,N′-((propane-2,2-diylbis(sulfanediyl))bis(ethane-2,1-diyl))diacrylamide, or disulfanediylbis(ethane-2,1-diyl) diacrylate. In some embodiments of the invention that may be mentioned herein, the cleavable crosslinking moiety may be derived from N,N′-Bis(acryloyl)cystamine or N,N′-((propane-2,2-diylbis(sulfanediyl))bis(ethane-2,1-diyl))diacrylamide.

As disclosed herein, the composite material may comprise constitutional units that comprise a moiety capable of covalently bonding to a polar molecule. For example, in some embodiments of the invention that may be mentioned herein, the composite material may comprise constitutional units that comprise a moiety having a functional group capable of reacting with a mixture of formaldehyde and tris(hydroxymethyl)aminomethane to form a functional group having the formula —CH₂—NHC(CH₂OH)₃. Examples of such groups that may be mentioned herein include a phenol ring (e.g. a tyrosine moiety) and catechol ring (e.g. a dopamine moiety or a 3,4-dihydroxybenzylamine moiety). A particular example that may be mentioned herein is a phenol ring (e.g. a tyrosine moiety).

In some embodiments of the invention that may be mentioned herein, the patterned hydrogel may comprise constitutional units comprising a moiety (e.g. an antibody) targeted to a biomolecule. For example, the constitutional units may comprise a moiety (e.g. an antibody) targeted to biomolecule selected from the group consisting of a protein biomarker, a DNA sequence, and an RNA sequence.

When the patterned hydrogel comprises constitutional units comprising a moiety (e.g. an antibody) targeted to a biomolecule, the moiety (e.g. an antibody) may be targeted to a biomolecule that recruits an enzyme that is capable of catalysing a crosslinking reaction and/or a cleavage reaction. This may advantageously provide for more localised crosslinking/cleavage of the hydrogel.

For example, in some embodiments of the invention that may be mentioned herein the biomolecule may recruit an enzyme selected from the group consisting of an oxidase enzyme, a peroxidase enzyme, a protease and an enzyme that cleaves DNA. In some embodiments of the invention that may be mentioned herein the biomolecule may recruit an enzyme selected from the group consisting of horseradish peroxidase (HRP), glucose oxidase, glutathione peroxidase, laccase, tyrosinase and glutathione reductase. In some embodiments of the invention that may be mentioned herein the biomolecule may recruit an enzyme selected from the group consisting of horseradish peroxidase (HRP), glucose oxidase and glutathione peroxidase.

In some embodiments of the invention that may be mentioned herein, the patterned hydrogel may comprise constitutional units comprising a moiety (e.g. an antibody) targeted to a biomolecule selected from the group consisting of CD63, CD24, EpCAM, EGFR, MUC1, CD125, HER2 and CEA. In some embodiments of the invention that may be mentioned herein, the patterned hydrogel may comprise constitutional units comprising a moiety (e.g. an antibody) targeted to a biomolecule selected from the group consisting of CD63, CD24, EpCAM, EGFR, MUC1 and CD125.

In some embodiments of the invention that may be mentioned herein, the one or more target molecule recognition moieties may comprise a crosslinkable moiety and/or a cleavable crosslinking moiety; and
the patterned hydrogel may comprise constitutional units comprising a bio-moiety that recruits an enzyme that is capable of catalysing a crosslinking reaction between the crosslinkable moieties, or a cleavage reaction of a cleavable crosslinking moiety, which crosslinking reaction or cleavage reaction imparts a stress value S₂ to the patterned hydrogel.

In some such embodiments of the invention that may be mentioned herein, the constitutional units comprising a crosslinkable moiety and/or a cleavable crosslinking moiety may comprise a redox-responsive moiety. Examples of specific redox-responsive moieties include a phenol moiety (e.g. a tyrosine moiety) and a thiol moiety.

In some embodiments of the invention that may be mentioned herein, the constitutional units comprising one or more target molecule recognition moieties may comprise constitutional units comprising a crosslinkable moiety selected from the group consisting of a phenol moiety (e.g. a tyrosine moiety) and a thiol moiety. In some such embodiments that may be mentioned herein, the constitutional units comprising or more target molecule recognition moieties may comprise constitutional units derived from one or more of the group consisting of N-acryloyltyramine (NATA), 2-mercaptoethyl acrylate and N-(2-mercaptoethyl)acrylamide (MEAM). In some such embodiments that may be mentioned herein, the constitutional units comprising or more target molecule recognition moieties may comprise constitutional units derived from one or more of the group consisting of N-acryloyltyramine (NATA) and N-(2-mercaptoethyl)acrylamide (MEAM).

In some embodiments of the invention that may be mentioned herein, the constitutional units comprising one or more target molecule recognition moieties may also comprise constitutional units comprising a cleavable crosslinking moiety selected from the group consisting of a disulfide moiety and a thioketal moiety. In some such embodiments that may be mentioned herein, the cleavable crosslinking moiety may be derived from N,N′-Bis(acryloyl)cystamine, N,N′-((propane-2,2-diylbis(sulfanediyl))bis(ethane-2,1-diyl))diacrylamide, or disulfanediylbis(ethane-2,1-diyl) diacrylate. In some such embodiments that may be mentioned herein, the cleavable crosslinking moiety may be derived from N,N′-Bis(acryloyl)cystamine, or N,N′-((propane-2,2-diylbis(sulfanediyl))bis(ethane-2,1-diyl))diacrylamide.

In some embodiments of the invention that may be mentioned herein, the composite material may comprise a stimulus-transmitting layer disposed between the substrate and the patterned hydrogel. The stimulus-transmitting layer may comprises a stimulus-transmitting material capable of transmitting a stimulus to the stimulus-responsive constitutional units, where the said stimulus-responsive constitutional units are responsive to said stimulus. This may help to amplify any effect generated by the stimulus-responsive constitutional units. In some such embodiments, the stimulus-transmitting layer may have a thickness of from 5 to 50 nm.

In some embodiments of the invention that may be mentioned herein, the stimulus-transmitting material may be selected from one or more of the group consisting of a thermally conductive material and an electrically conductive material, such as thermally conductive material. In some such embodiments, the thermally conductive material may be a photothermally conductive material, such as a photothermally conductive material configured to apply a thermal stimulus to the stimulus-responsive constitutional units upon plasmonic heating of the photothermally conductive material.

In some embodiments of the invention that may be mentioned herein, the thermally conductive material may be selected from one or more of the group consisting of gold, silver, copper, aluminium, CuₓS, platinum and zinc (e.g. gold). In some embodiments of the invention that may be mentioned herein the thermally conductive material may be selected from one or more of the group consisting of gold, silver, copper, aluminium, and CuₓS (e.g. gold).

In some embodiments of the invention that may be mentioned herein, the stimulus-responsive constitutional units may be selected from one or more of the group consisting of thermally-responsive constitutional units, electrically-responsive constitutional units, optically-responsive constitutional units, magnetic-responsive constitutional units and pH-responsive constitutional units. In some such embodiments, the stimulus-responsive constitutional units may be selected from one or more of the group consisting of thermally-responsive constitutional units and pH-responsive constitutional units. Examples of specific thermally-responsive constitutional units that may be mentioned herein include thermally-responsive constitutional units formed from one or more of the group consisting of N-isopropylacrylamide (NIPAM), di(ethylene glycol)methylether methacrylate (DEGMA), triethylene glycol acrylate (TEGA), N-vinylcaprolactam (NVCL) and N-ethyl-N-methylacrylamide (EMA). Particular examples of specific thermally-responsive constitutional units that may be mentioned herein include thermally-responsive constitutional units formed from one or more of the group consisting of N-isopropylacrylamide (NIPAM), di(ethylene glycol)methylether methacrylate (DEGMA), triethylene glycol acrylate (TEGA) and N-vinylcaprolactam (NVCL). Examples of specific pH-responsive constitutional units that may be mentioned herein include pH-responsive constitutional units formed from acrylic acid (AA), methacrylic acid (MAA), 4-vinylbenzoic acid (VBA), 2-(demethylamino)ethyl methacrylate (DMAEMA), 2-(diethylamino)ethyl methacrylate (DEAEMA), 2-vinylpyridine (2VP), 11-acrylamidoundecanoic acid (AaU) and sodium 2-acrylamido-2-methylpropanesulfonate (AMPS), such as acrylic acid.

In some embodiments of the invention that may be mentioned herein, the patterned hydrogel may be patterned to have a lattice structure, for example a lattice comprising substantially square-shaped holes.

In some embodiments of the invention that may be mentioned herein, the reversible buckling and/or reversible swelling of the patterned hydrogel may be detectable by scanning electron microscopy and/or laser diffraction. In some such embodiments, the patterned hydrogel may comprises left-handed and/or right-handed structures.

The invention also provides a method of detecting a biomolecule target in a sample, comprising the steps:
(i) providing a composite material according to the invention as described herein, and a source of a stimulus to which the stimulus-responsive material is responsive;
(ii) applying the stimulus at a first magnitude to the composite material in the presence of said sample;
(iii) repeating step (ii) at a different stimulus magnitude to determine the threshold stress level T;
(iv) contacting the composite material with a sample and simultaneously applying the stimulus at a magnitude for which:

$${S}_{1} < T and {S}_{1} + {S}_{2} > T , or$$

$${S}_{1} > T and {S}_{1} + {S}_{2} < T ;$$

(v) determining the presence of said biomolecule target upon detecting a conformational change (e.g. change in buckling state) of the patterned hydrogel, wherein
   a change in buckling state indicates the presence of said biomolecule target.

In some embodiments of the invention, the source of a stimulus to which the stimulus-responsive material is responsive may be a source of thermal energy or a pH change. For example, the source may be a source of thermal energy that is a source of electromagnetic radiation. In some such embodiments, irradiation of a stimulus-transmitting material, when present, by the source of thermal energy (e.g. a source of electromagnetic radiation) may provide plasmonic heating of the stimulus-responsive material.

As discussed herein, in some embodiments of the invention the method may comprise determining the buckling and/or swelling of the patterned hydrogel using scanning electron microscopy (SEM). In some embodiments of the invention the method may comprise determining the buckling and/or swelling of the patterned hydrogel using laser diffraction. In some such embodiments, the patterned hydrogel may comprise left-handed and/or right-handed structures.

In some embodiments of the invention that may be mentioned herein, the one or more target molecule recognition moieties may comprise crosslinkable moieties, and the patterned hydrogel may comprise constitutional units comprising a moiety (e.g. an antibody) targeted to a biomolecule that recruits an enzyme that is capable of catalysing a crosslinking reaction of the crosslinkable moieties. In some such embodiments, the enzyme may be selected from the group consisting of an oxidase enzyme, a peroxidase enzyme, a protease and an enzyme that cleaves DNA, for example selected from the group consisting of horseradish peroxidase (HRP), glucose oxidase, and glutathione peroxidase.

The invention is illustrated in more detail in the below Examples.

### EXAMPLES

### Materials

All chemicals were purchased from commercial vendors and used for synthesis without further purification, unless otherwise indicated. 2-hydroxyethyl acrylate, N-isopropylacrylamidee and ethylene glycol dimethylacrylate were purchased from Sigma Aldrich. CD63 antibody, LAMP-1, Flotillin 1, TSG101 and horseradish peroxidase (HRP)-conjugated streptavidin were purchased from BD Biosciences. N-succinimidyl acrylate was purchased from TCI Chemical. Zeba spin column, radio-immunoprecipitation assay (RIPA) buffer containing protease inhibitors, bicinchoninic acid (BCA) assay, ELISA plates, chemiluminescent substrate (SuperSignal West Pico PLUS) and blocking agents (SuperBlock) were purchased from Thermo Scientific. Polydimethylsiloxane (PDMS) was purchased from Dow Corning. Hydrogen peroxide (H₂O₂) solution was purchased from Thermo Fisher. Polyvinylidene fluoride membrane (PVDF) was purchased from Invitrogen. Dulbecco's modified Eagle's medium (DMEM) and RPMI-1640 medium were purchased from Hyclone. Fetal bovine serum (FBS) and penicillin-streptomycin were purchased from Gibco. MycoAlert Mycoplasma Detection Kit (LT07-418) was purchased from Lonza. Alix, HSP90 and HRP-conjugated secondary antibody were purchased from Cell Signaling. HSP70 was purchased from BioLegend.

TABLE 1
Protein markers and antibodies used.

| Protein | | |
|---|---|---|
| marker | Description | Antibody |
| CD63 | A type III lysosomal membrane protein | BD |
| | abundant and characteristic in exosomes. | Biosciences, |
| | | clone H5C6 |
| CD24 | A small heavily glycosylated cell adhesion | eBioscience, |
| | molecule, expressed in hematological | clone SN3 |
| | malignancies and solid tumors. | A5-2H10 |
| EpCAM | Epithelial cell adhesion molecule, | R&D Systems, |
| | transmembrane glycoprotein expressed | clone 158206 |
| | exclusively in epithelial and epithelial | |
| | neoplasms. | |
| MUC1 | Mucin 1, a cell surface glycoprotein and cell | Fitzgerald, |
| | adhesion molecule lining stomach, intestines, | clone |
| | and several other organs. | M01102909 |

### Analytical Techniques

### Nuclear Magnetic Resonance (NMR) Spectroscopy

NMR spectroscopy was carried out using Bruker 400 MHz or 500 MHz NMR spectrometer.

### General Procedure for Statistical Analysis

Unless otherwise stated, all measurements were performed in at least triplicate, and the data displayed as mean±standard deviation. Correlation analysis was performed with linear regression to determine the goodness of fit (R²). Significance tests were performed via a two-tailed Student's t-test. For inter-sample comparisons, multiple pairs of samples were each tested, and the resulting P values were adjusted for multiple hypothesis testing using Bonferroni correction. Values that had an adjusted P<0.05 were determined as significant.

### General Procedure for Cell Culture

All human cancer cell lines were obtained from American Type Culture Collection. HCT116, DLD-1, A431 and GL136 were grown in DMEM supplemented with 10% FBS and 1% penicillin-streptomycin. MKN45, SNU484, H3255 and PC9 were cultured in RPMI-1640 medium supplemented with 10% FBS and 1% penicillin-streptomycin. All cell lines were tested and free of mycoplasma contamination (MycoAlert Mycoplasma Detection Kit, LT07-418).

### Example 1. Synthesis of Hydrogel Monomers

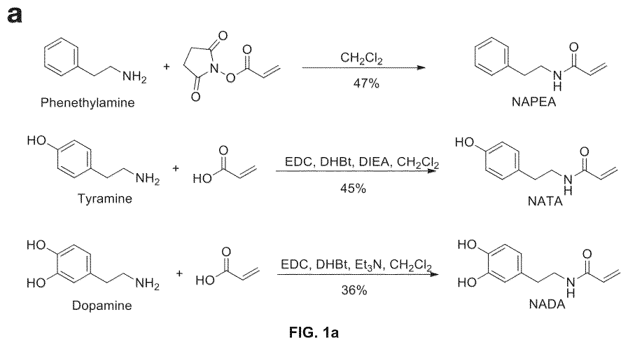

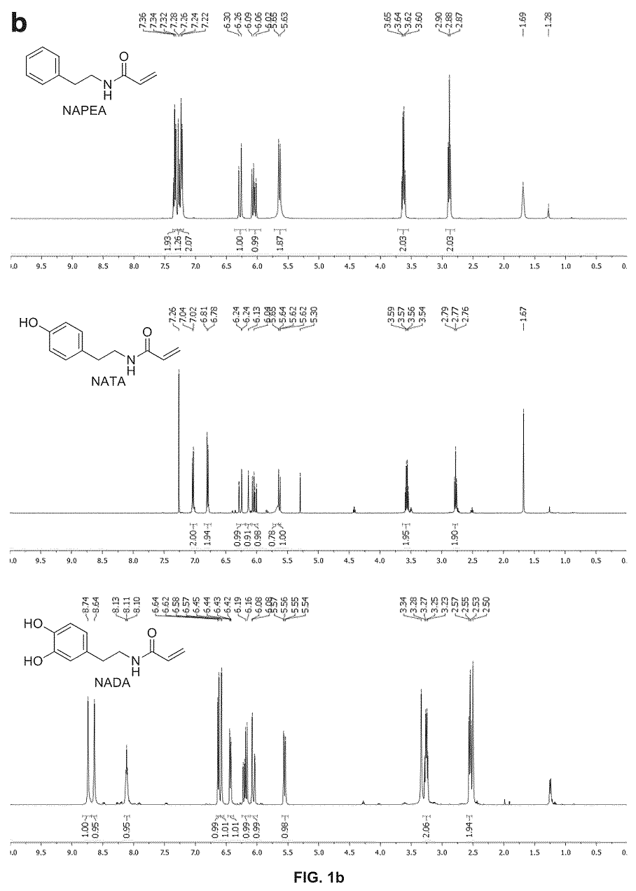

Hydrogel monomers were synthesized through direct acryloylation (FIG. 1a).

### Nata

To a solution of 1-ethyl-3-(3-dimethylaminopropyl)carbodiimide hydrochloride (EDC.HCl; 1 g, 5.2 mmol) in CH₂Cl₂ (20 mL), acrylic acid (0.25 mL, 3.6 mmol), 3,4-dihydro-3-hydroxy-4-oxo-1,2,3-benzotriazine (DHBt; 120 mg, 0.72 mmol) and N,N-diisopropylethylamine (DIEA; 0.63 mL, 3.6 mmol) were added in an ice bath. The mixture was stirred at 0° C. for 5 min before the addition of tyramine (500 mg, 3.6 mmol) and DIEA (0.63 mL). The reaction was then stirred at room temperature overnight, before being diluted with CH₂Cl₂ (30 mL) and washed with water. The aqueous layers were extracted with CH₂Cl₂ and the combined organics were washed with brine and dried over Na₂SO₄. Sticky precipitate formed was also collected by dissolving in methanol. Upon the addition of silica gel and evaporation, it was purified by silica gel chromatography (MeOH:CH₂Cl₂=8%) to afford the desired product as a colorless oil (310 mg, 45%) and characterized through ¹H NMR.

¹H NMR (400 MHz, CDCl₃) δ 7.03 (d, J=8.5 Hz, 2H), 6.79 (d, J=8.5 Hz, 2H), 6.26 (dd, J=17.0, 1.3 Hz, 1H), 6.13 (s, 1H), 6.04 (dd, J=17.0, 10.3 Hz, 1H), 5.74-5.60 (br, 1H), 5.63 (dd, J=10.3, 1.3 Hz, 1H), 3.56 (dd, J=13.0, 7.0 Hz, 2H), 2.77 (t, J=7.0 Hz, 2H).

### Napea

To a solution of phenethylamine (0.25 mL, 2 mmol) in CH₂Cl₂ (20 mL), N-acryloxysuccinimide (338 mg, 2 mmol) was added to the solution in an ice bath. The mixture was allowed to slowly warm up and stirred at room temperature overnight. Upon the addition of silica gel and evaporation, the reaction was directly purified by silica gel chromatography (ethyl acetate (EtOAc):Hexane (Hex)=1:1) to afford the desired product as a colorless oil (165 mg, 47%).

¹H NMR (400 MHz, CDCl₃) δ 7.34 (t, J=7.4 Hz, 2H), 7.27 (d, J=9.2 Hz, 1H), 7.23 (d, J=7.5 Hz, 2H), 6.28 (d, J=17.0 Hz, 1H), 6.05 (dd, J=17.0, 10.3 Hz, 1H), 5.73-5.54 (br, 1H), 5.64 (d, J=10.3 Hz, 1H), 3.63 (q, J=6.6 Hz, 2H), 2.88 (t, J=6.9 Hz, 2H).

### NADA

EDC.HCl (1 g, 5.2 mmol) in CH₂Cl₂ (20 mL), acrylic acid (0.25 mL, 3.6 mmol), DHBt (120 mg, 0.72 mmol) and Et₃N (0.50 mL, 3.6 mmol) were added in an ice bath. The mixture was stirred at 0° C. for 5 min before the addition of dopamine hydrochloride (690 mg, 3.6 mmol) and Et₃N (0.50 mL). The reaction was then stirred at room temperature overnight, before being diluted with CH₂Cl₂ (30 mL) and washed with water. The aqueous layers were extracted with CH₂Cl₂ and the combined organics were washed with brine and dried over Na₂SO₄. Sticky precipitate formed was collected by dissolving in methanol. Upon the addition of silica gel and evaporation, it was purified by silica gel chromatography (MeOH:CH₂Cl₂=10%) to afford the desired product as a colorless oil (275 mg, 36%).

¹H NMR (400 MHz, dimethyl sulfoxide (DMSO)) δ 8.74 (s, 1H), 8.64 (s, 1H), 8.11 (t, J=5.2 Hz, 1H), 6.63 (d, J=7.9 Hz, 1H), 6.58 (d, J=1.6 Hz, 1H), 6.43 (dd, J=7.9, 1.6 Hz, 1H), 6.19 (dd, J=17.1, 10.0 Hz, 1H), 6.06 (dd, J=17.1, 2.2 Hz, 1H), 5.55 (dd, J=10.0, 2.2 Hz, 1H), 3.25 (dd, J=14.1, 6.5 Hz, 2H), 2.54 (t, J=7.5 Hz, 2H).

### Example 2. Preparation of Dual-Responsive Hydrogel Precursor

### Preparation of Antibody CD63-Acrylate Monomer

To prepare the antibody monomer, CD63 antibody (100 μL, 0.5 mg/mL in phosphate buffered saline (PBS)) was mixed with 5 μL of N-succinimidyl acrylate (200 mM DMSO stock). The reaction was incubated for 1 h at room temperature, before being desalted through a Zeba spin column.

### Preparation of Dual-Responsive Hydrogel Precursor

Using the synthesized monomers in Example 1, we prepared a dual-responsive hydrogel that can be cross-linked in response to temperature stimulus and free radicals. The addition of antibody monomer further confers molecular recognition. Specifically, to form the hydrogel, we first prepared the precursor mixture: NIPAM was dissolved in 2-hydroxyethyl acrylate (HEA) monomer at a mass ratio of 1:1, before the addition of NATA monomer (25 mg/mL) and CD63-acrylate monomer (prepared above, 5.0 μg/mL). Ethylene glycol dimethylacrylate (EGDMA, 20 mg/mL) and 2-hydroxy-2-methyl-1-phenyl-propan-1-one (Darocur 1173, 25 mg/mL) were subsequently added as the cross-linker and photoinitiator, respectively. Leveraging the hydrogel properties, we used the antibody specificity from CD63-acrylate to achieve target capture, the thermal responsiveness from NIPAM to achieve critical locking and the cross-linking ability of NATA, in the presence of free radicals, to achieve molecular measurements.

### Example 3. Mechanical Metamaterial Operating at Critical Point for Hyper-Responsive Analysis (MORPH) Platform

Hydrogel-based mechanical metamaterials present unique opportunities in achieving dramatic bio-responsiveness; they can be readily engineered, through tailoring their materials composition and structured geometry, to transduce and amplify even faint biomolecular interactions. Nevertheless, several challenges remain to realize such potential. Firstly, these metamaterials have a narrow window of dramatic responsiveness (i.e., at the critical transition state), which may be easily missed due to intrinsic variabilities during hydrogel casting. Secondly, as most hydrogels rely on bulk target diffusion within the gel matrix to actuate, they are slow to respond and lack the ability to distinguish spatial distribution of stimuli. The MORPH technology is designed to address both challenges, through critical-tuning and amplified transduction of mechanical metamaterials, to enable hyper-responsive and informative molecular analysis.

To address these challenges, we developed a versatile analytical platform that leverages the advanced behaviors of mechanical metamaterials for nanoscale molecular profiling. Named MORPH, this technology employs a dual-responsive, hydrogel-based mechanical metamaterial as a shape-transforming chiral interferometer. Specifically, the MORPH platform is prepared in a hyper-responsive state (the critical transition state) through plasmonic thermal modulation of the cured hydrogel metamaterial to maximize its mechanical strain while preserving the patterned geometry. The platform can thus be activated by even sparse biomolecular stimuli; these stimuli readily perturb the critically-strained metamaterial and trigger a chiral reorganization of the metamaterial geometry to induce amplified optical diffraction.

### Preparation of PDMS Molds

To pattern the hydrogel as a mechanical metamaterial, we prepared PDMS molds. Through standard soft-lithography processing, a 15 μm-thick cast mold was fabricated with SU-8 photoresist and silicon wafers using a cleanroom mask aligner (SUSS MicroTec), and developed after UV exposure. Subsequently, PDMS and cross-linker were mixed at a ratio of 10:1, casted onto the fabricated SU-8 mold, and cured at 75° C. overnight to form the PDMS mold. The PDMS mold has a periodic lattice of square-holes. The square hole is 25 μm×25 μm, and the periodicity (the distance between the centres of adjacent holes) is 50 μm.

### Hydrogel Patterning (Hydrogel Mechanical Metamaterial)

To enable plasmonic locking of the hydrogel metamaterial, we deposited thin films of titanium (Ti, 3 nm) and gold (Au, 10 nm) onto a glass wafer, using an electron beam evaporator (AJA ATC-2030-E HV), and further modified the substrate surface with N,N′-Bis(acryloyl)cystamine solution (1 μg/mL) for 3 h. Finally, to pattern the hydrogel, we added 2 μL of the freshly prepared hydrogel precursor (prepared in Example 2) onto the Au-coated glass substrate, and covered the solution with the PDMS mold prepared above. After hydrogel bonding and curing through UV exposure, the PDMS mold was peeled off.

### SR Characterization

To characterize the hydrogel swelling properties at the microscale, a hydrogel pillar was patterned through PDMS molding (as described in the protocol for the preparation of PDMS molds above). The formed hydrogel was then immersed in deionized water and observed under a microscope (Leica DMi8) to measure volumetric changes in situ. The SR was calculated based on the equation:

$$SR = ( V - {V}_{0} ) / {V}_{0} * 100 \% \qquad ( 2 )$$

where V is the wet volume and V₀ is the dry volume.

To characterize the hydrogel swelling properties at the macroscale, 100 μL of hydrogel precursor (prepared in Example 2) was cured under UV exposure (385 nm, 2 min). The formed hydrogel was peeled off and its dry mass was measured. After immersion in deionized water, its wet mass was measured. The SR was calculated based on the equation:

$$SR = ( m - {m}_{0} ) / {m}_{0} * 100 \% \qquad ( 3 )$$

where m is the wet mass and m₀ is the dry mass.

### Plasmonic Locking for Amplified Response

To critically-tune the hydrogel mechanical metamaterial, we utilized plasmon-induced localized heating to modulate and stabilize hydrogel swelling. All plasmonic locking was performed after hydrogel patterning and casting, through contactless LED illumination. Briefly, we first established the relationship between the DI and the SR by scanning the LED injection current from 0 to 500 mA at a step of 20 mA. With this relationship, we determined the optimal locking state (P_{lock}) for various types of stimulus-induced deformation changes. For stimulus-induced swelling changes, the locking state was chosen as the critical SR (critical point); for stimulus-induced cross-linking changes, the locking state was chosen as the SR that is 5% larger than the critical point. Using the corresponding LED injection current, we set the metamaterial to its optimized locking state. To experimentally validate the approach, we induced swelling changes by applying tromethamine (25 mM) and formaldehyde (25 mM) in PBS (pH=6.5) for 5 min. Likewise, we induced cross-linking changes by applying HRP (1 μg/mL) and H₂O₂ (3%) for 5 min.

### SEM

Different-state metamaterials were prepared separately. Metamaterial in the breathing state was frozen at −80° C. for 30 min and then dried in a freeze dryer (Labconco 4.5) overnight. Metamaterial in the buckling state was fully swollen in deionized water before the drying process. After coating with a 5-nm gold layer using a sputter coater (Polalis), the samples were imaged with a SEM (FEI Verios 460).

### Microfluidic Device Fabrication

To facilitate plasmonic locking and fluid flow over the metamaterial, a microfluidic device comprising three layers was prototyped. The bottom layer (substrate layer) housed the hydrogel metamaterial, which was patterned on Au-coated glass substrate as described above. Onto this substrate layer, a microfluidic layer was constructed using a tabletop CO₂ laser engraver (Universal) and assembled through silicone-based adhesive (Adhesives Research), to incorporate fluidic channels and reaction chambers. Finally, a cover layer comprising laser-cutter PMMA was aligned and bonded to the microfluidic layer to include inlets and outlets. As depicted in FIG. 2, there are steps:
1. clean silicon water;
2. pattern SU-8 photoresist;
3. develop;
4. mould PDMS;
5. deposit Au film on glass;
6. pattern hydrogel with PDMS mold; and
7. bond with microfluidic chip.

### MORPH Optical Measurement (Smartphone)

To enable point-of-care analysis, we further developed a smartphone-based sensor that comprises seven components: a 3D-printed optical cage, a LED source, a laser diode, a cube beamsplitter, an optical filter, a magnification lens and a driving circuit. The optical cage was printed with a desktop 3D printer (Ultimaker 3) and included four easily-assembled parts to hold different components of the smartphone-based sensor. The low-cost beam-splitter was used to combined the laser source (OSRAM, λ=520 nm) incident from its left and the LED source (CZR S&T, λ=440 nm) incident from its bottom. The optical filter (cut-on λ=500 nm) and magnification lens (f=7.7 mm) were used to improve the imaging quality. The driving circuit was used to modulate the LED output power. The assembled system measured 85 mm (length)×50 mm (width)×60 mm (height) in dimension and was equipped with two sliding slots for quick attachment to smartphones (Apple). The images were recorded and analyzed through a smartphone interface with the same analysis approach, as in the customized imaging system in Example 9. Sensor performance was evaluated against a commercial microplate reader (Tecan) for different fluorescent dyes and intensities.

### Results and Discussion

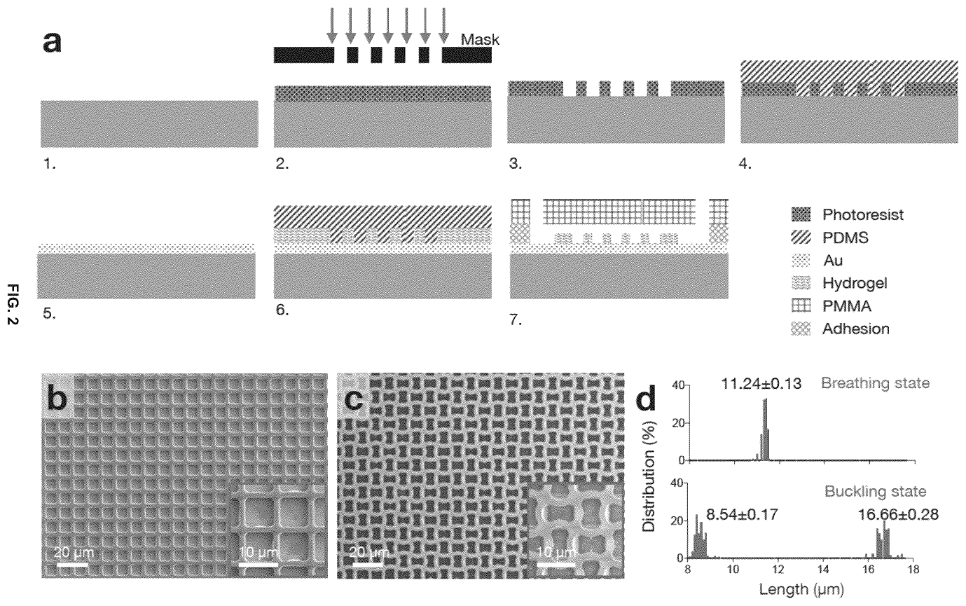

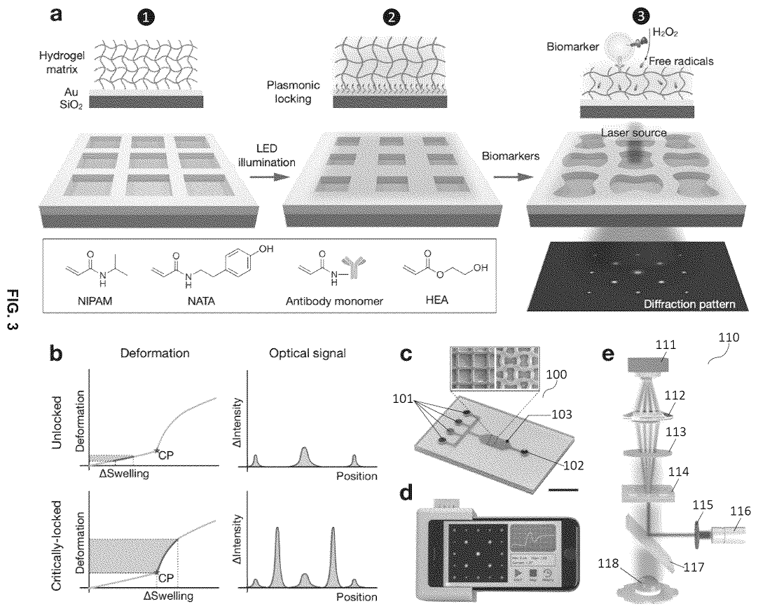

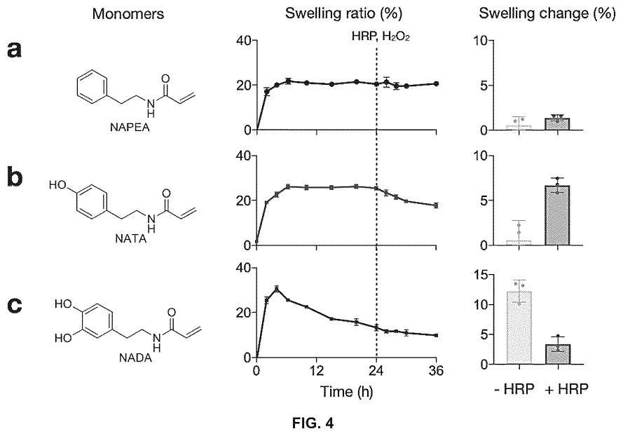

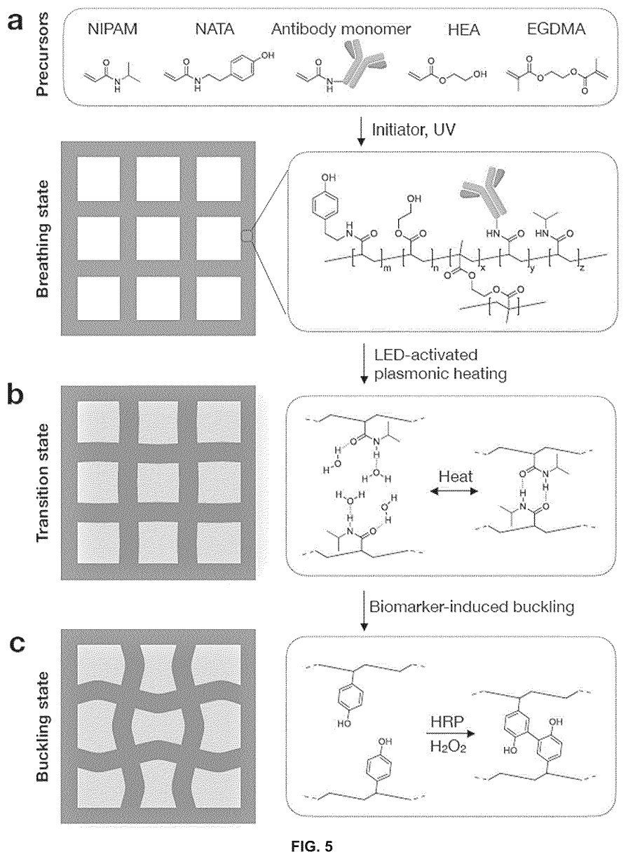

The MORPH platform is designed to boost the hydrogel's responsiveness to biomolecular stimuli. It features a tunable mechanical metamaterial that is patterned in a dual-responsive hydrogel (i.e. temperature and redox activity) and also serves as an optical interferometric mask. Through critical modulation, the metamaterial mask is tuned to a hyper-responsive state that can readily respond to biomolecules and change its patterned geometry to induce optical diffraction changes (FIG. 3a). The MORPH workflow thus comprises three functional steps: metamaterial patterning, critical-point locking and target-induced pattern transformation. During metamaterial casting, the hydrogel matrix (comprising NIPAM as the temperature-responsive monomer and NATA as the redox-responsive monomer (FIGS. 1 and 4)) was organized as a regular array of crossed, elastic beams (i.e. to form a periodic lattice of square-holes) (FIG. 5a) and covalently patterned onto a gold-coated glass substrate (FIG. 2a). The incorporation of antibody monomers into the hydrogel network confers molecular specificity to capture and concentrate target biomolecules onto the metamaterial. In the next step, through LED-activated plasmonic heating at the gold-hydrogel interface (Son, J. H. et al., Light Sci. Appl. 2015, 4, e280-e280; and Han, F., Soeriyadi, A. H. & Gooding, J. J., Macromol. Rapid Commun. 2018, 39, e1800451), the metamaterial was precisely tuned to its critical point, a hyper-responsive transition state that is between its breathing and buckling states (FIG. 5b). Finally, biomolecules were immuno-captured onto the metamaterial; in the presence of specific biomarkers, free radicals are generated through antibody-peroxidase activity to induce fast and localized hydrogel cross-linking. This mechanical perturbation breaks the transition state swiftly, leading to a cooperative, chiral transformation of the metamaterial pattern (FIGS. 5c and 2b-d); such transformation can be detected in real time through changes in the projected diffraction pattern to inform about the biomarker composition.

In comparison to its unlocked state (e.g. breathing state), the critically-locked metamaterial is designed to detect scarce biomolecules, by generating amplified deformations and optical signals. When incubated with a low concentration of biomarkers (i.e. target-induced swelling change is small), the metamaterial in its breathing state experiences only minimal, linear deformations; the biomarker-induced perturbation is insufficient to trigger a pattern transformation and thus causes only small changes in the optical diffraction pattern (FIG. 3b, top). In comparison, when the metamaterial is critically-locked (near its critical point), an equal amount of biomarker-induced perturbation triggers a rapid release of the accumulated strain energy, to induce a dramatic, nonlinear pattern transformation of the metamaterial (to its buckling state). This geometric reorganization causes a distinct mode change in the projected diffraction pattern, thereby enabling amplified optical detection of scarce biomolecules (FIG. 3b, bottom).

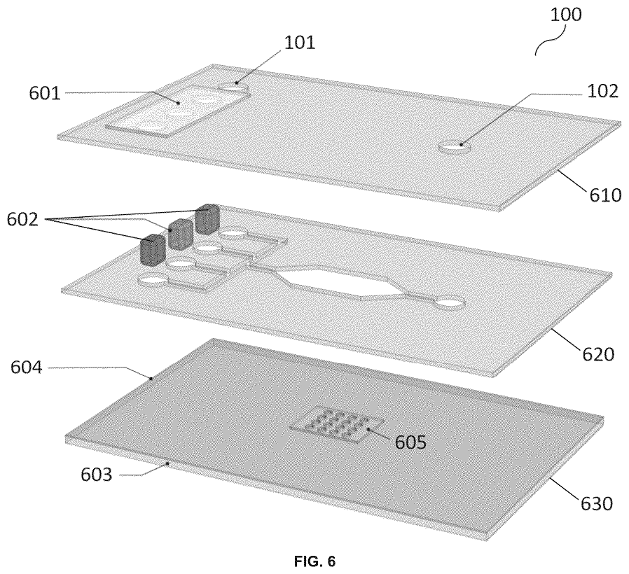

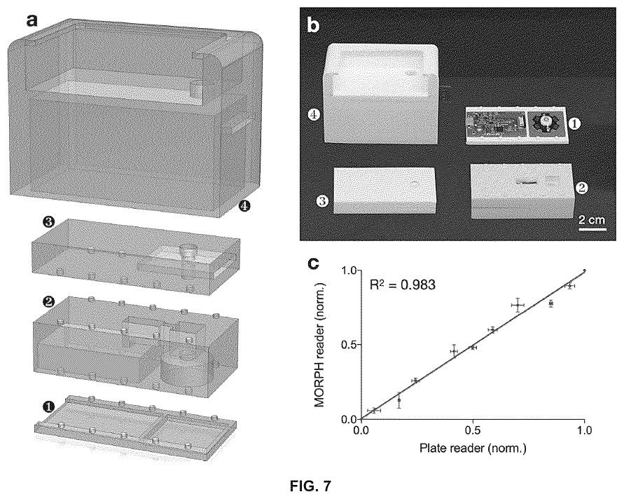

To facilitate MORPH molecular profiling in complex clinical biofluids, we implemented the technology in a hydrogel/PMMA hybrid microfluidic system (FIG. 3c). As depicted in FIG. 3c, the microfluidic device 100 includes inlets 101, an outlet 102 and a reaction chamber 103. The device 100 not only achieves on-chip critical-locking of the mechanical metamaterial, but also streamlines the MORPH assay workflow (FIG. 6). As depicted in FIG. 6, the device 100 includes a cover layer 610 having an adhesive sealing tape 601, inlets 101 and an outlet 102, a microchannel layer 620 having preloaded reagents 602, and a substrate layer 630 having a glass 603, a plasmonic heating Au film 604 and a mechanical metamaterial 605. Furthermore, the microfluidic system can be loaded onto a custom-designed, smartphone-based optical detector 110 to enable real-time interferometric measurements for kinetic analysis (FIGS. 3d-e and 7). As depicted in FIG. 3e, the smartphone-based optical detector 110 includes a smartphone camera 111, lens 112, a filter 113, a chip 114, a pinhole 115, a laser diode 116, a beamsplitter 117 and a LED 118. Image acquisition and data analysis could be achieved automatically through a smartphone interface.

### Example 4. Critical Point in Pattern Transformation

To investigate the critical point in a pattern transformation, we first studied the mechanical metamaterial deformation as a function of hydrogel swelling. Using the patterned hydrogel metamaterial (i.e. a periodic array of square-holes, prepared in Example 3), we experimentally modulated its swelling through temperature control.

### Optical Simulation and Metamaterial Design

To optimize the hydrogel metamaterial design, so as to maximize chiral interferometric detection, we performed full 3D finite-difference time-domain (FDTD) simulations using a commercial software package (FDTD Solutions, Lumerical). An infinitely large metamaterial pattern was modeled as a unit cell with periodic boundary conditions. Each unit cell comprises two clockwise-rotating and two counterclockwise-rotating cross-structures. Each cross-structure has a dimension of 15 μm (length)×5 μm (width)×15 μm (thickness). The refractive indices of the hydrogel structure and the surrounding medium were set to 1.4560 and 1.33, respectively. A uniform mesh of 5 nm was applied in all directions. The structure was illuminated with a plane wave from the top and the transmitted electromagnetic field was recorded by a monitor placed 0.5 μm beneath the structure. The rotation angle was increased from 0° to 45° in steps of 1°. The recorded near-field information was then projected to the far-field to obtain the diffraction patterns. By establishing the relationship between the diffraction patterns, rotation angles and deformation indices, we thus optimized the design of mechanical metamaterial structure to achieve maximal optical response.

### Numerical Simulation of Metamaterial Buckling

Metamaterial deformation was simulated using a nonlinear finite element analysis software (ABAQUS/Standard). A two dimensional array (11×11) of square holes (length: 10 μm, periodicity: 15 μm) was embedded in a square sheet with a dimension of 180 μm (length)×180 μm (width)×15 μm (thickness). Each mesh was composed of 15-node, quadratic, hybrid, 3D elements (ABAQUS element type C3D15H). The elastomeric stress-strain behavior was modeled as an incompressible neo-Hookean solid with a shear modulus of 0.5 MPa (Musgrave, C. S. A. & Fang, F., Materials 2019, 12, 261). A z-axis constraint was applied to the bottom surface. Compression loads were applied to the four sidewalls in respective perpendicular directions. An eigenvalue buckling analysis was first conducted to determine the buckling mode. The mode-shaped geometric imperfection was then introduced into the subsequent perturbation steps of the nonlinear buckling analysis.

### Results and Discussion

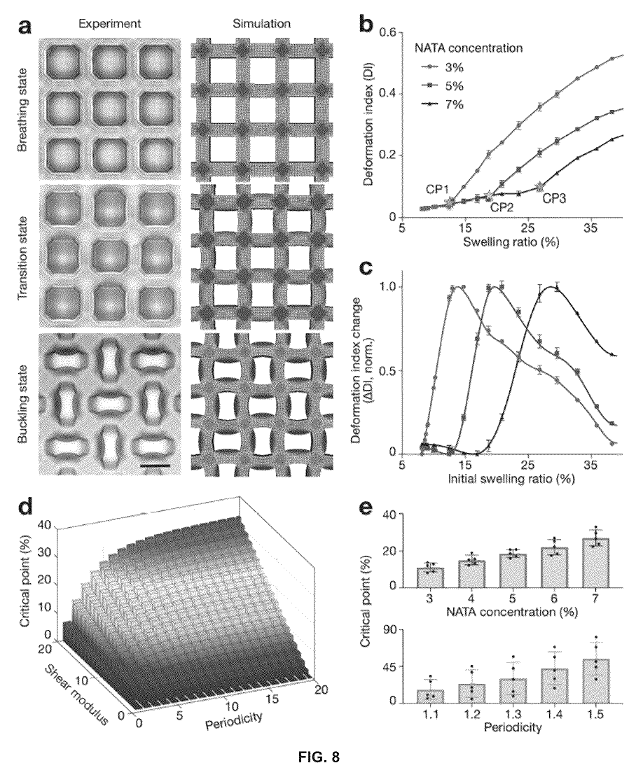

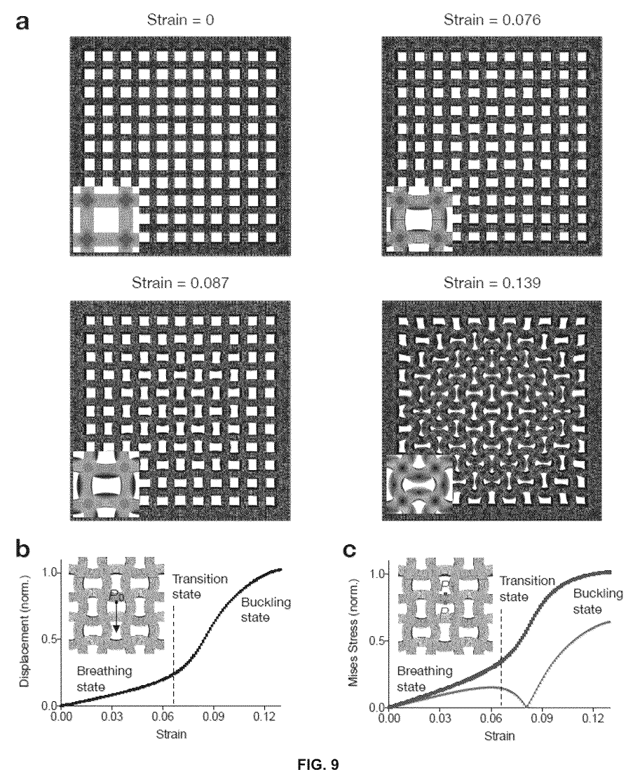

With increasing swelling, the metamaterial demonstrated geometric changes; it preserved its square-hole morphology (breathing state to transition state) before it rapidly buckled to form mutually orthogonal rectangular holes (buckling state) (FIG. 8a, left). We further validated this pattern transformation through nonlinear finite element simulation (FIGS. 8a, right and 9a). The simulation results indicate that the rate of metamaterial deformation increased sevenfold when the pattern transformation was triggered (FIG. 9b). Stress distribution analysis further demonstrated that the accumulated stress was suddenly released and converted into mechanical deformation at the critical point (FIG. 9c).

### Example 5. Relationship of Pattern Transformation with Hydrogel Swelling

To characterize the relationship of pattern transformation with hydrogel swelling, we defined the SR to reflect volumetric changes during hydrogel swelling and the deformation index to characterize geometric changes (of a unit cell in the metamaterial) during pattern transformation.

### Deformation Index Characterization

To characterize geometric changes, the hydrogel metamaterial was imaged with a microscope (Leica DMi8). Through image analysis (ImageJ), the dimensions of a unit cell were measured. The DI was calculated based on the equation:

$$DI = \sqrt{\frac{1}{2} {( \frac{L - {L}_{0}}{{L}_{0}} )}^{2} + \frac{1}{2} {( \frac{W - {W}_{0}}{{W}_{0}} )}^{2}} \qquad ( 4 )$$

where L₀ and W₀ denote the original length and width of the unit cell; L and W are the length and width after geometric changes.

### Results and Discussion

The results are shown in FIG. 10. We measured the deformation curve as a function of the SR for a range of hydrogel compositions (FIG. 8b). The experimental results agree well with the simulation prediction: across all compositions tested, at a small SR, the metamaterial preserved its geometry and the deformation index increased minimally and linearly; at a threshold SR (critical point, CP), the metamaterial showed an abrupt increase in geometric deformation upon further swelling. Differences in the respective critical points (e.g., CP1 vs. CP2) could be attributed to variations in hydrogel compositions and properties (Tanaka, T. & Fillmore, D. J., J. Chem. Phys. 1979, 70, 1214-1218). Based on these measurements, we assessed the first derivate of the deformation curves, to evaluate deformation changes induced by a fixed increment in SR (5%, a typical swelling change observed in bio-responsive hydrogels, Miyata, T., Uragami, T. & Nakamae, K., Adv. Drug Deliv. Rev. 2002, 54, 79-98). The results confirm that the mechanical metamaterial experienced the most amplified deformation changes when its initial SR was precisely matched to its critical point (FIG. 8c).

### Example 6. Various Hydrogel Factors that can Determine the Setting of the Critical Point

To exploit the critical point for amplifying deformation changes, we next investigated various hydrogel factors that can determine the setting of the critical point. These pre-casting factors, namely the hydrogel's intrinsic mechanical property (shear modulus) and structural geometry (periodicity), can be experimentally adjusted through the hydrogel composition and mask design, respectively (i.e. before hydrogel casting).

### Adjustment of Hydrogel Composition

The preparation of various hydrogel compositions was carried out by following the preparation of dual-responsive hydrogel precursor protocol in Example 2 except the doped NATA monomer concentration was changed from 3% to 7%, at an interval of 1%.

### Adjustment of Mask Design

The preparation of mask was performed by following the protocol in Example 3. The mask design was adjusted by changing the periodicity of the square hole array from 1.1 to 1.5, at an interval of 0.1.

### Results and Discussion

Through theoretical modelling (Cai, S. et al., Soft Matter 2010, 6, 5770), we observed that the critical point increases with increasing shear modulus and/or periodicity (i.e. a larger swelling change is needed to trigger the buckling of stiffer gels and stubbier structures) (FIG. 8d). The simulation results were experimentally validated, where we increased the hydrogel's shear modulus (through doping with NATA monomer) and changed the periodicity of the casting mold (FIG. 8e). Notably, these pre-casting tuning approaches showed considerable variations even among technical replicates (FIG. 8e). This not only indicates that critical points are delicate states, but also highlights the experimental challenges in critical-tuning solely through these pre-casting factors.

### Example 7. Plasmonic Locking of Critical Point

To address the challenges mentioned in Example 6, we developed a post-curing strategy (i.e., after hydrogel casting) to precisely tune and lock metamaterials. Plasmonic locking experiments were carried out by following the plasmonic locking protocol in Example 3. Numerical simulation of metamaterial buckling was carried out by following the numerical simulation of metamaterial buckling protocol in Example 4.

### Results and Discussion

We applied plasmonic heating to control the temperature of the casted hydrogel (FIG. 11a). LED illumination was applied directly to the metamaterial to activate surface plasmon resonance at the gold-hydrogel interface (Son, J. H. et al., Light Sci. Appl. 2015, 4, e280-e280; and Xin, H., Namgung, B. & Lee, L. P., Nat. Rev. Mater. 2018, 3, 228-243); through controlled plasmonic heating, the temperature-responsive hydrogel metamaterial experiences a distinct swelling strain and is critically-locked to a desired swelling state.

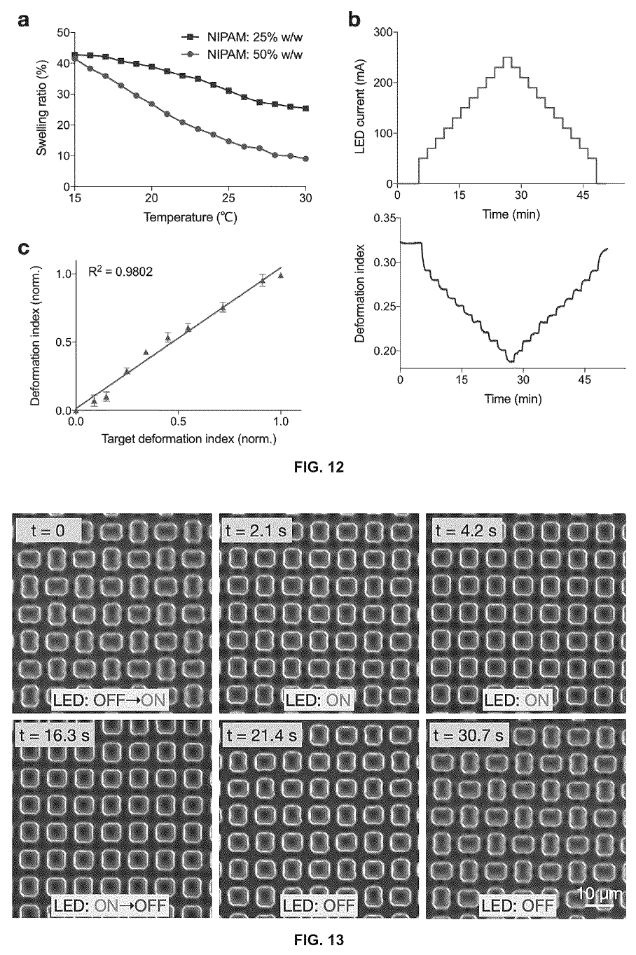

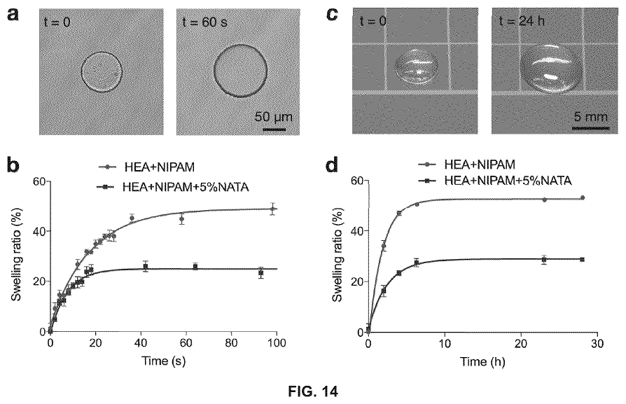

To optimize this plasmonic modulation, we measured the absorption spectra for a range of gold-film thickness (FIG. 11b) and thus chose the 10 nm gold film for good overall transmission, a 440 nm LED source for plasmonic locking (enhanced absorption), and a 520 nm laser source for subsequent MORPH interferometric measurements (enhanced transmission). Using this experimental configuration, we next investigated the kinetics and spatial distribution of plasmonic heating. Through numerical simulations, we found that heat generated at the gold layer could be rapidly transferred throughout the hydrogel (FIG. 11c). Using the optimized gel composition (50% w/w NIPAM), which yielded a wide range of thermal modulation (FIG. 12a), we experimentally validated the finding; the patterned metamaterial showed a rapid, repeatable deformation response to plasmonic modulation (FIG. 11d). The slower experimental response (˜16.3 s) (FIG. 13), in comparison to the simulation result (˜100 ms), could be attributed to the hydrogel's inherent swelling kinetics at different size scales (FIG. 14). By adjusting the LED current to modulate the plasmonic activity, we achieved precise and versatile tuning of the metamaterial deformation (FIGS. 12b-c).

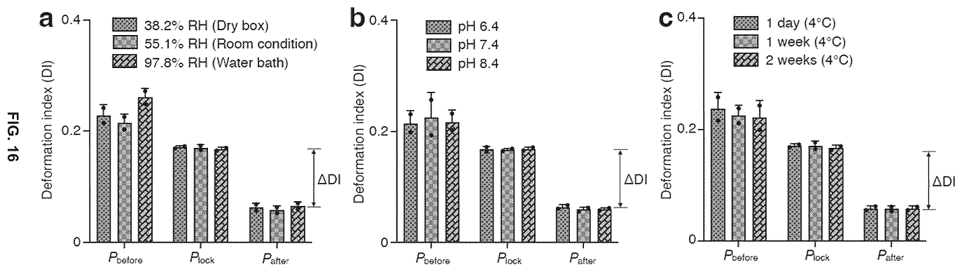

We next evaluated if the approach can be applied to amplify different types of deformation changes (i.e. stimulus-induced hydrogel swelling vs. cross-linking) (FIG. 15a). Specifically, to exploit the amplified deformation responses near the critical point, we adapted the plasmonic tuning to lock the metamaterial to respective swelling states (P_{lock}), so as to accommodate different stimulus-induced responses (FIG. 11e). To amplify stimulus-induced swelling changes, by applying a large LED current (I_{LED}), we tuned and locked the metamaterial from its initial state (P_{before}) to its critical point (P_{lock}). Subsequent application of the stimulus (formaldehyde and tromethamine) caused the hydrogel metamaterial to swell to its final state (P_{after}) and achieved a large increase in the deformation index. To amplify stimulus-induced cross-linking changes, we critically tuned the metamaterial with a reduced LED current (from P_{before} to P_{lock}) such that further cross-linking resulted in a highly contracted final state (P_{after}, also the critical point) and thus a large decrease in the DI. As compared to matched systems without plasmonic locking (FIG. 15b), the critically-locked platforms demonstrated not only precise tuning (across different initial states), but also broad applicability to amplify various types of stimulus-induced deformation changes (up to 69.4-fold enhancement) (FIG. 11f). Finally, we verified the effectiveness of the critical modulation to compensate for various external factors during device storage, namely humidity, pH, and storage duration at 4° C. (FIG. 16). Although the MORPH devices showed variable initial states (P_{before}) upon different storage conditions, they could be effectively tuned to the critical states (P_{lock}) and thus showed a similar stimulus-induced response (ΔDI).

### Example 8. Interferometric Projection of Metamaterial Deformation

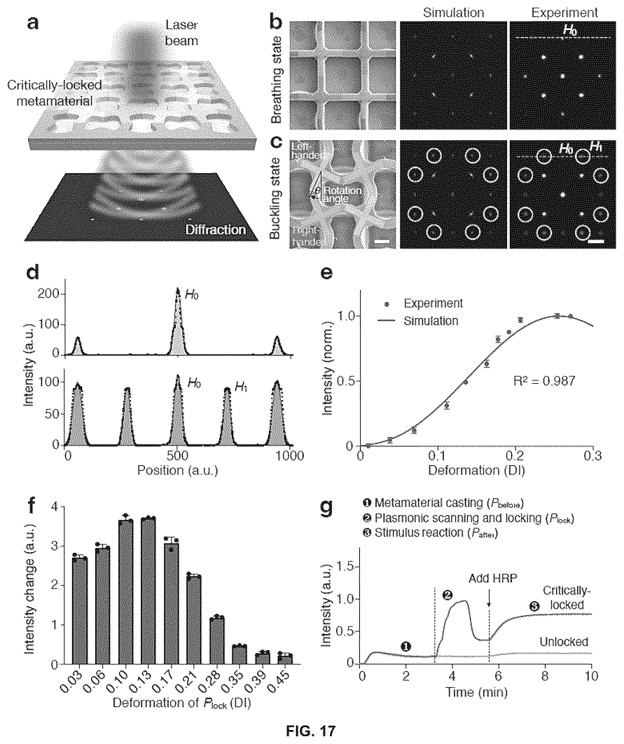

Next, we applied the critically-tuned metamaterial as a chiral interferometer. We evaluated the MORPH pattern transformation by measuring its projected light diffraction (FIG. 17a). Plasmonic locking experiments were carried out by following the plasmonic locking protocol in Example 3. Numerical simulation was carried out by following the numerical simulation of metamaterial buckling protocol in Example 4.

### Measurement of Projected Light Diffraction

The laser beam passing through hydrogel mechanical metamaterials was focused by a convex lens, and then collected by a monochrome CCD camera. The software interface of the CCD camera was used to capture diffraction images.

### Results and Discussion

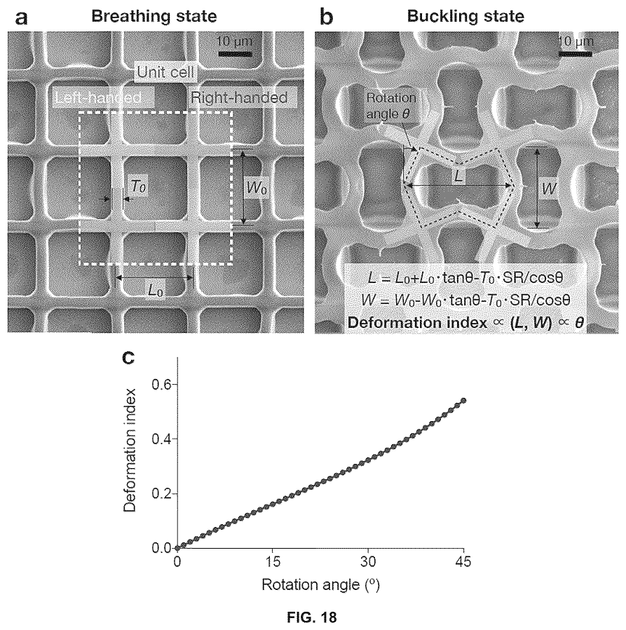

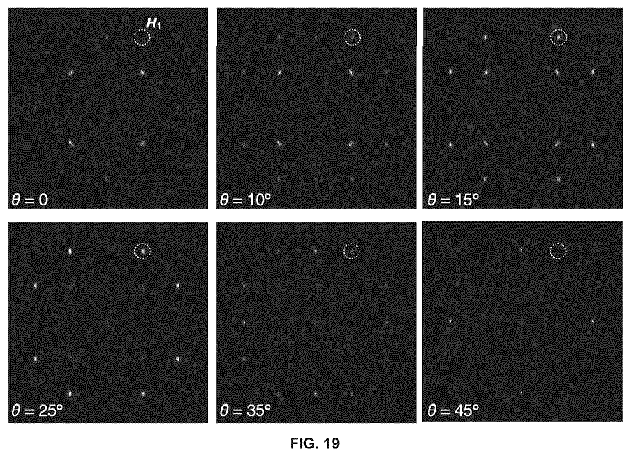

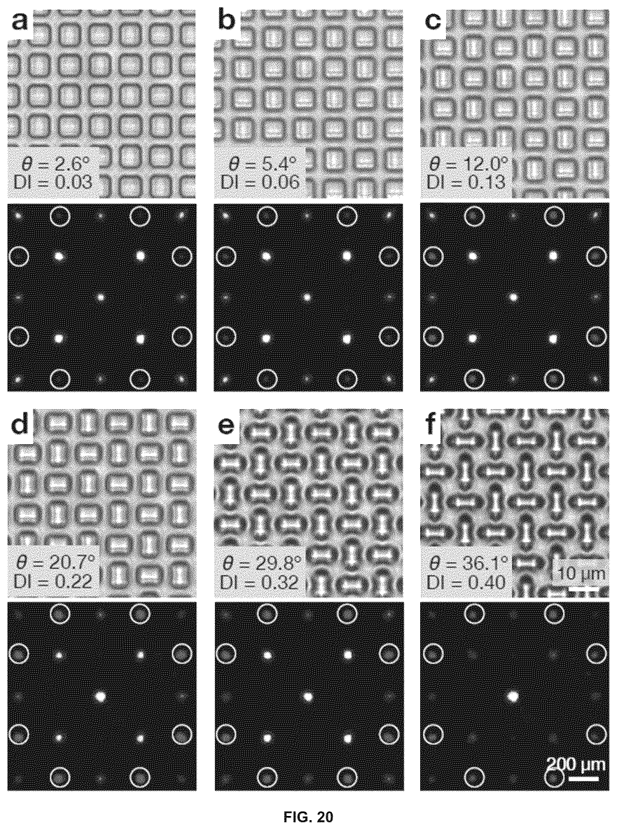

Specifically, we exploited the metamaterial mask as a 2D array of handed cross-structures; the chirality of adjacent cross-structures (left-handed vs. right-handed) was determined by their angled rotation upon pattern transformation (FIGS. 18a-b). Increasing hydrogel swelling could thus be resolved as a continuous function of metamaterial deformation, represented either as the DI or as the chiral rotation (FIG. 18c). In the breathing state (FIG. 17b, left), the hydrogel metamaterial experienced minimal deformation, thereby preserving the spatial relationship between adjacent opposite-handed structures. In this case, the metamaterial functions as two orthogonally-placed diffraction gratings (Palmer, E. W. et al., Rep. Prog. Phys. 1975, 38, 975-1048). As determined by both numerical simulation and experimental validation, this mask pattern resulted in a classic 2D diffraction pattern (FIG. 17b). In the buckling state, after pattern transformation, adjacent left-handed and right-handed structures rotate in opposite directions to form a chiral interferometer (FIG. 17c, left). Light beams passing through the two opposite-handed structures interfered with each other to create a distinct diffraction pattern (FIG. 17c). As compared to that of the breathing state, the projected diffraction pattern by the buckled metamaterial showed new optical hotspots (FIG. 17d). Numerical simulations (FIG. 19) and experimental results (FIG. 20) confirm that the optical intensities of these emerging hotspots strongly correlated to the corresponding metamaterial deformations (FIG. 17e, R²=0.987).

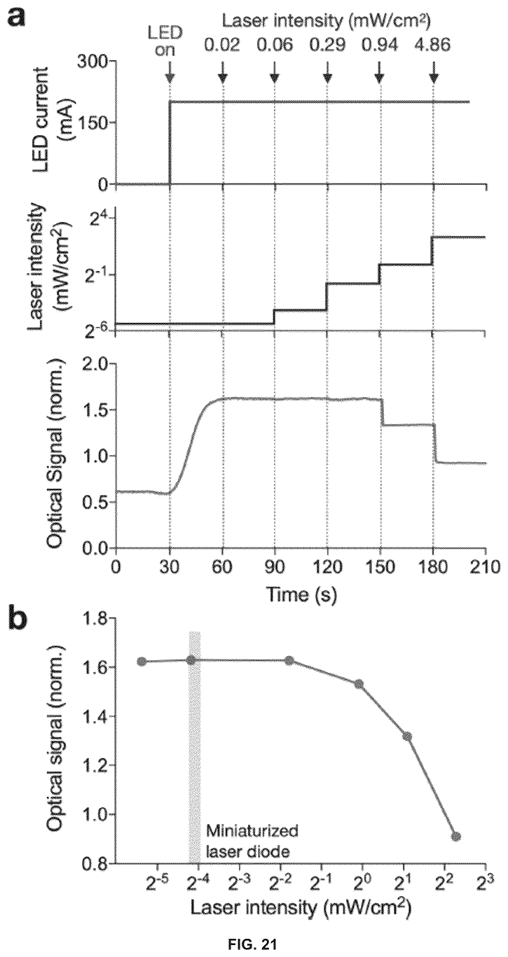

We subsequently validated these emerging diffraction hotspots in for real-time measurements of biological stimuli (i.e., peroxidase-generated free radicals for hydrogel cross-linking). We first confirmed the robustness of plasmonic locking in amplifying chiral interferometric measurements. Little interference was observed between plasmonic locking (through LED excitation) and interferometric measurements (through laser transmission) (FIG. 21). We next tuned the metamaterial through plasmonic modulation, so as to determine the optimized locking state (P_{lock}) for maximal optical signal. With the applied stimulus, the system demonstrated a maximal intensity change when the deformation index was carefully set (FIG. 17f, P_{lock}=0.13). As demonstrated by real-time optical sensorgrams, when locked to this optimized condition, the platform not only demonstrated rapid response, but also amplified the optical signal (FIG. 17g).

### Example 9. MORPH for Clinical Exosome Profiling

We finally developed the MORPH platform for informative exosome profiling in native clinical biofluids. An attractive circulating biomarker, exosomes are nanoscale extracellular membrane vesicles (30-200 nm in diameter) actively secreted by cells into the circulation. They abound in biofluids and carry reflective molecular cargos.

### ELISA

Anti-CD63 capture antibodies (5 μg/mL) were adsorbed onto ELISA plates and blocked in PBS containing 1% bovine serum albumin (BSA) for 2 h before incubation with samples. After washing with PBST (PBS with 0.05% Tween 20), biotinylated detection antibodies (e.g. anti-CD63, anti-CD24, anti-EpCAM, and anti-MUC1, at 1 μg/mL) were added and incubated for 2 h at room temperature. Following incubation with HRP-conjugated streptavidin and chemiluminescent substrate, chemiluminescence intensity was determined (Tecan).

### Vesicle Mixture Preparation

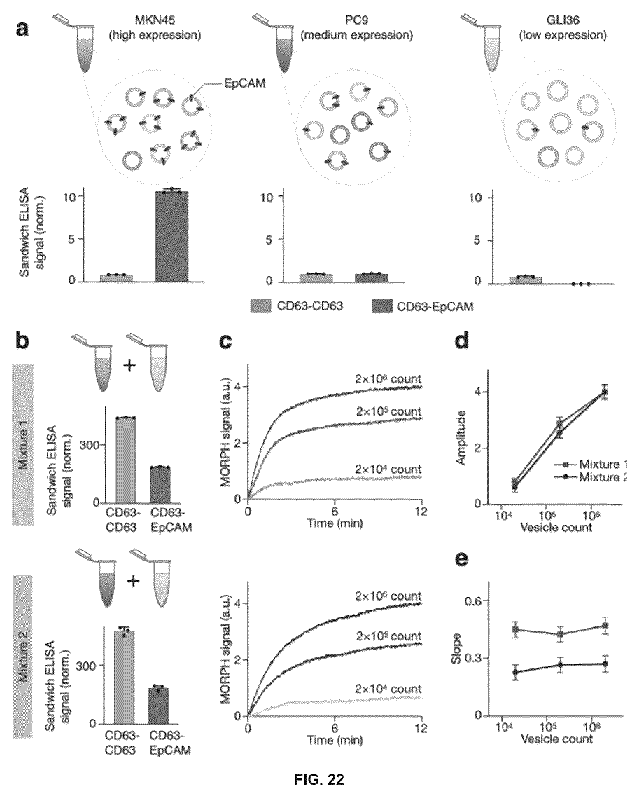

To evaluate the real-time MORPH measurement, we prepared complex vesicle mixtures with comparable total biomarker abundance but different biomarker distribution. Using vesicles derived from single cell lines, which are known to differentially expressed EpCAM, we profiled their vesicular expression using the gold-standard ELISA (through CD63 capture and EpCAM detection). When vesicle count-matched, these singly-derived vesicle solutions showed a similar CD63 signal but a different EpCAM abundance (MKN45, high; PC9, medium; GLI36, low). Next, we combined these singly-derived vesicle solutions to prepare complex vesicle mixtures. Specifically, we prepared Mixture 1 by adding the high-expression MKN45 exosome solution with the low expression GLI36 exosome solution, and Mixture 2 by adding the medium-expression PC9 exosome solution with the low expression GL136 exosome solution (FIG. 22). These vesicle mixtures were adjusted to match in vesicle counts. On ELISA measurement, both complex mixtures showed indistinguishable profiles, indicating that they contained a similar amount of total biomarker, despite their different vesicle composition.

### Exosome Isolation and Quantification

Cells at passages 1-15 were cultured in vesicle-depleted medium (containing 5% vesicle-depleted fetal bovine serum, dFBS) for 48 h before vesicle collection. All media containing extracellular vesicles were filtered through a 0.2-μm membrane filter (Millipore), isolated by differential centrifugation (first at 10,000 g and subsequently at 100,000 g). For independent quantification of vesicle concentration, we used the NTA system (NS300, Nanosight). Vesicle concentrations were adjusted to obtain ˜50 vesicles in the field of view to achieve optimal counting. All NTA measurements were done with identical system settings for consistency.

### MORPH Workflow for Exosome Molecular Profiling

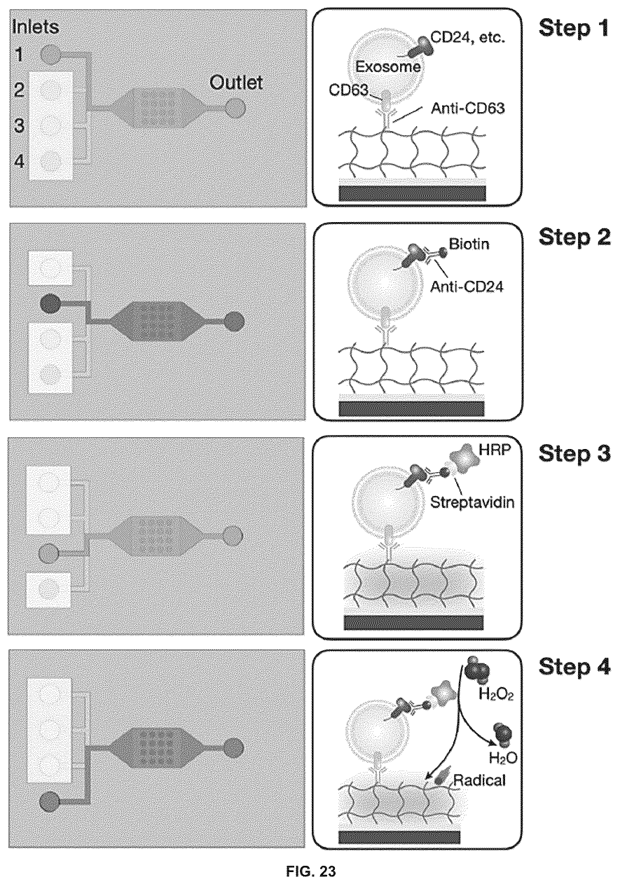

MOPRH microfluidic devices (prepared in Example 3) were extensively treated with blocking agents (SuperBlock) during device storage. Before MORPH application, we flushed each device with PBS and applied plasmonic modulation to critically tune and maintain the metamaterial for subsequent measurement (see above for details). During measurement, sample solution (5 μL) was introduced into the MORPH device and incubated on the critically-locked metamaterial for 5 min, to enable specific vesicle capture through anti-CD63 antibody. Sample-matched control was performed through a critically-locked metamaterial functionalized with IgG isotope control antibody (by following the same protocol for anti-CD63 antibody) to account for nonspecific vesicle binding. To establish a sandwich configuration, the immobilized vesicles were incubated with biotinylated detection antibodies (e.g. anti-CD63, anti-CD24, anti-EpCAM, and anti-MUC1, 1 μg/mL) for 3 min, before being washed and treated with HRP-conjugated streptavidin (1 μg/mL) for 1 min. After washing, H₂O₂ solution (3%) was introduced for 1 min. Solution introduction was actuated by a syringe pump; solution incubation was performed at a flow rate of 1 μL/min and washing was performed at a flow rate of 10 μL/min. Only in the presence of antibody-HRP complex, free radicals were generated through HRP catalysis to cross-link the hydrogel. Optical signals were recorded in real time (see MORPH optical measurement protocol below). Details on the antibodies used are listed in Table 1. The operation of the MORPH platform is also depicted in FIG. 23.

As depicted in FIG. 23, the operation of the MORPH platform includes 4 steps:

### Step 1: Exosome Capture

Inlets 2-4 are sealed with adhesive tape.
Inlet 1 is applied with PBS to hydrate the hydrogel metamaterial, followed by clinical samples for exosome capture;

### Step 2: Addition of Biotinylated Antibodies

Biotinylated antibodies (e.g. anti-CD63 and anti-CD24), preloaded at inlet 2, are introduced to the reaction chamber.
Unbound antibodies are removed through flushing;

### Step 3: Addition of HRP-Conjugated Streptavidin

HRP-conjugated streptavidin, preloaded at inlet 3, is introduced to the reaction chamber.
Unbound molecules are removed.
The metamaterial is critically-locked, through plasmonic modulation; and

### Step 4: Metamaterial Cross-Linking

Hydrogen peroxide solution, preloaded at inlet 4, is introduced. Free radicals are catalyzed by the immobilized HRP.
Free radicals induce metamaterial cross-linking.

### MORPH Optical Measurement (Customized Imaging System)

Interferometric measurements were performed on a customized imaging system. A collimated laser (Thorlabs LP520-SF15, λ=520 nm) and a LED diode (CZR S&T, λ=440 nm) were used as the probe source for diffraction pattern imaging and the modulation source for critical point locking, respectively. The two light sources were combined using a cube beamsplitters (Thorlabs BS007) and then illuminated on the microfluidic chip. Subsequently, the diffracted light passed through a long-pass filter (cut-on λ=500 nm, Thorlabs FEL0500) and a lens (f=25 mm, Thorlabs LB1014) before being captured by a CMOS camera (Nikon DS-Fi3). Continuous, real-time measurements were recorded using a commercial software (NIS-Elements D) and analyzed in ImageJ.

### MORPH Optical Analysis

For MORPH analysis, we plotted the mean intensity of the hotspots of interest as a function of time, and fitted the experimental data to the following equation:

$$I ( t ) = {I}_{max} \cdot [ 1 - exp ( - {K}_{M O R P H} \cdot t ) ] \qquad ( 5 )$$

where l(t) is the signal intensity at time t, Iₘₐₓ is the intensity maximum, and K_{MORPH} is a constant describing the kinetic response and is affected by the local concentration of biomarker-induced, short-lived free radicals.

We then characterized the following parameters:

End-point (amplitude) analysis reflects the maximum intensity and is defined:

$$amplitude = {I}_{max} \qquad ( 6 )$$

Kinetic (slope) analysis is defined as the rate of intensity change at half intensity maximum:

$$slope = \frac{1}{{I}_{max}} \cdot \frac{{I}_{max / 2}}{{T}_{max / 2}} = \frac{1}{2 \cdot {T}_{max / 2}} \qquad ( 7 )$$

where Iₘₐₓ is the intensity maximum, I_{max/2} is the half intensity maximum, and T_{max/2} is the time to reach the half intensity maximum.

By combining equation (5) and equation (7), slope=K_{MORPH}/ln(4). Therefore, we applied the slope analysis to characterize local changes in biomarker concentration and differentiate vesicle mixtures with a similar total biomarker abundance but different biomarker distribution.

### Western Blotting

Exosomes isolated by ultracentrifugation were lysed in radio-immunoprecipitation assay (RIPA) buffer containing protease inhibitors and quantified using BCA assay. Protein lysates were resolved by sodium dodecyl sulfate polyacrylamide gel electrophoresis (SDS-PAGE), transferred onto PVDF, and immunoblotted with antibodies against protein markers: CD63, LAMP-1, Alix, HSP90, HSP70, Flotillin 1, and TSG101. Following incubation with HRP-conjugated secondary antibody, enhanced chemiluminescence was used for immunodetection (Thermo Scientific).

### Results and Discussion

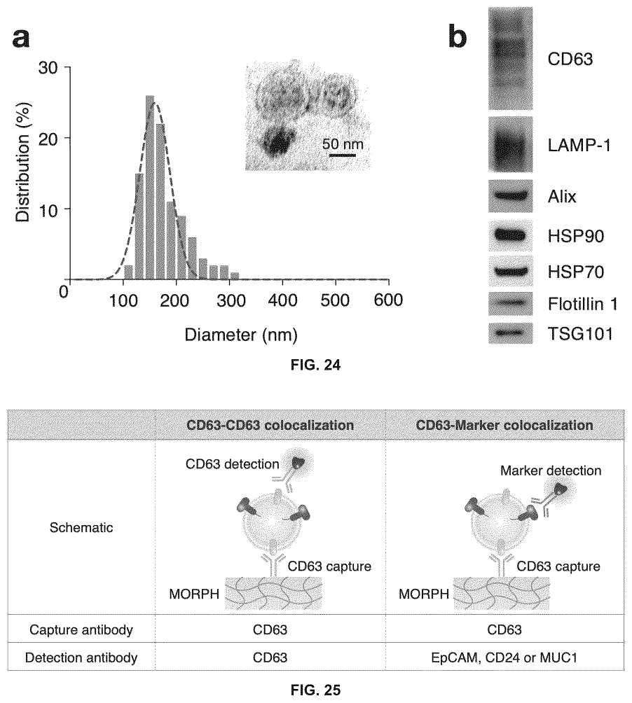

To evaluate biomarker signatures in exosomes (e.g. composition and distribution), we functionalized the MORPH metamaterial with antibodies against CD63, a type Ill lysosomal membrane protein commonly found in and characteristic of exosomes, to enrich vesicles onto the metamaterial (FIG. 24). The captured vesicles were then incubated with various detection antibodies (e.g. EpCAM) to form a sandwich configuration for biomarker co-localization study (FIG. 25). All detection antibodies were conjugated with HRP, which generates short-lived free radicals to induce rapid, localized metamaterial cross-linkings (FIG. 23).

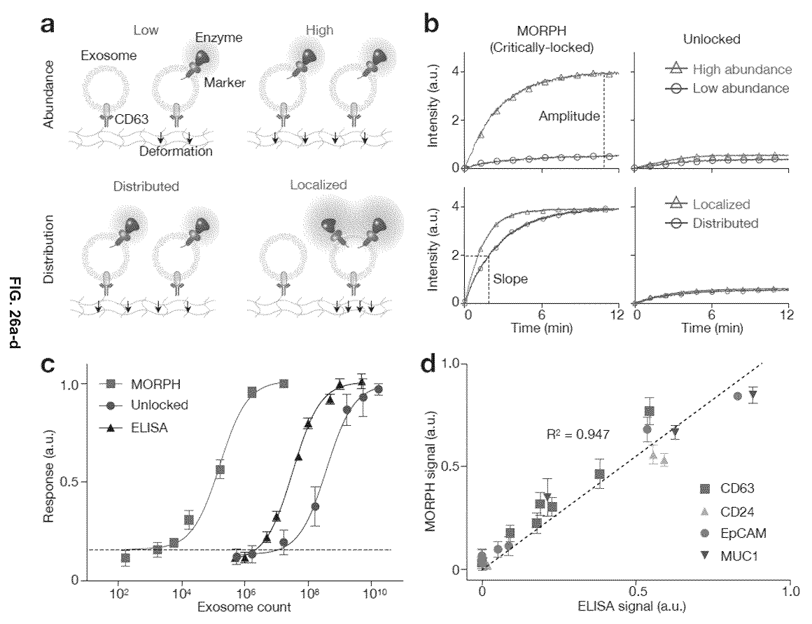

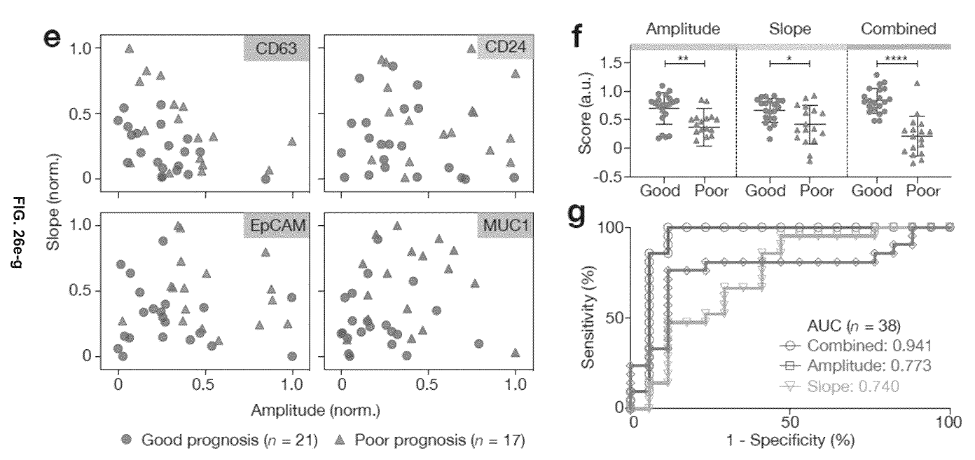

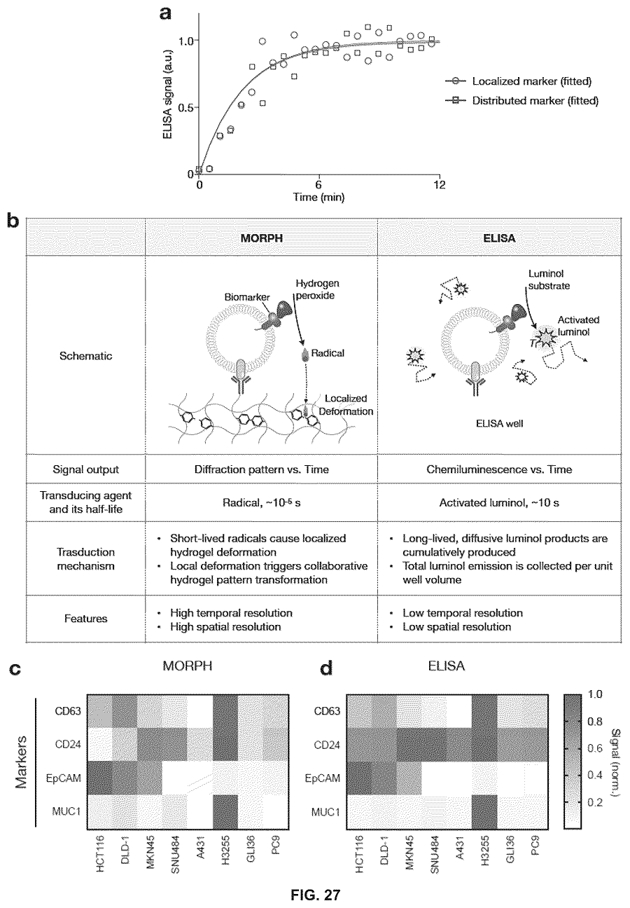

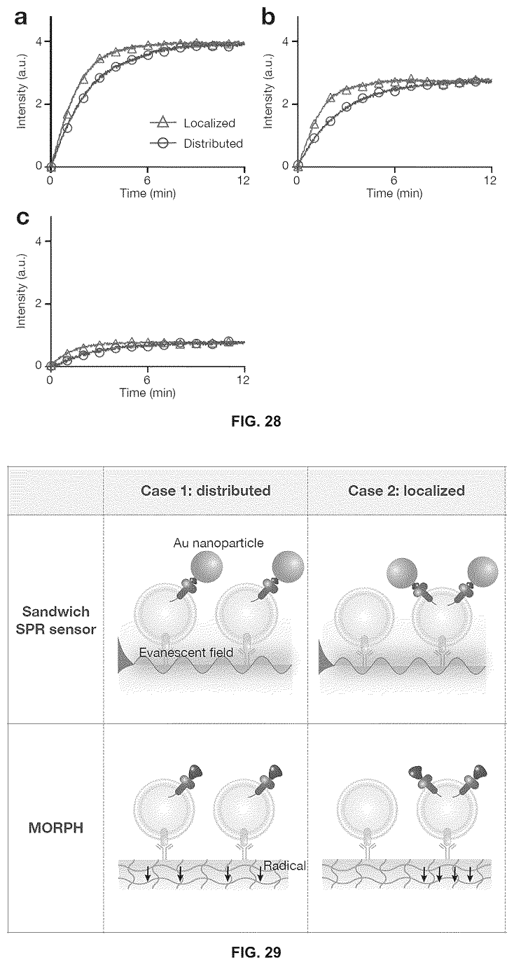

We first evaluated the MORPH technology in vesicle mixtures that express different total biomarker abundance and/or distribution states (FIG. 26a). Using the MORPH platform, we employed both end-point (amplitude) and kinetic (slope) analyses to characterize exosome mixtures (FIG. 26b). We defined amplitude as the intensity maximum, to measure the saturation MORPH intensity, and slope as the normalized rate of intensity change at half intensity maximum, to measure the speed of MORPH signal change. We next prepared complex vesicle mixtures to reflect different biomarker abundance and/or distribution states. Specifically, we mixed vesicles derived from different cell origins, known to have different vesicular biomarker expression (biomarker density per vesicle) (FIG. 22a), to prepare mixtures with a comparable total biomarker abundance but different vesicle composition (i.e. different biomarker distribution) (FIG. 22b). On conventional ELISA analysis, these complex mixtures showed indistinguishable profiles (FIG. 27a). We attribute this to the limited spatial and temporal resolution of ELISA measurement (FIG. 27b). For example, chemiluminescence ELISA measures the abundance of luminol products (over time); as these luminol products have a long half-life (˜10 s, Hohman, J. R. et al., Tetrahedron Lett. 1996, 37, 8273-8276) and experience extensive diffusion before measurement, ELISA kinetic measurements are temporally- and spatially-averaged and do not preserve biomarker spatial distribution. Real-time MORPH measurements, however, could distinguish these mixtures. The MORPH amplitude analysis correlated with total biomarker abundance while the slope analysis distinguished mixtures with different distribution states (FIGS. 22c-e). Across different tested mixtures, we observed a faster MORPH kinetic response (slope) for vesicle mixtures that contained more densely-localized biomarkers (FIG. 28). We attribute these observations to MORPH's high spatial resolution as compared to bulk measurements by ELISA and SPR sensors (Table 2 and FIG. 29). Specifically, MORPH preserves and encodes the spatial distribution of biomarkers as short-lived radicals. Due to their short half-life (˜10⁻⁵ s, Liu, M. et al., Nat. Commun. 2013, 4, 2029), these radicals have limited diffusion and react rapidly (i.e. only near the site of production); a high local concentration of radicals, generated by vesicles with densely-expressed biomarkers, causes efficient and localized hydrogel deformation (Carey, F. A. & Sundberg, R. J. Advanced Organic Chemistry (Springer Science & Business Media, 2007)). This results in a high local mechanical stress that readily breaks the buckling threshold to trigger a collaborative hydrogel pattern transformation, characteristic of mechanical metamaterials (Bertoldi, K. et al., Nat. Rev. Mater. 2017, 2, 17066; and Foucard, L. C. et al., Nonlinearity 2015, 28, 89-112). A localized biomarker distribution thus exhibits a faster reaction kinetics (steeper slope) than a distributed biomarker distribution.

TABLE 2
Comparison of MORPH and SPR sensors
with reference to FIG. 29.

| | Features |
|---|---|
| Sandwich | SPR sensor converts biomarker information into |
| SPR | refractive index changes (e.g. through binding of Au |
| sensor | nanoparticles) |
| | Measures bulk refractive index changes within the entire |
| | evanescent field |
| | High temporal resolution but low spatial resolution |
| | Similar kinetic profiles (amplitude and slope) for Cases 1 |
| | (distributed) and 2 (localized) |
| MORPH | MORPH converts biomarker information into short-lived |
| | radicals that cause localized hydrogel deformation; this |
| | accelerates collaborative hydrogel pattern transformation |
| | Distinguishes enhanced hydrogel pattern transformation |
| | High temporal and spatial resolution |
| | Different kinetic profiles (same amplitude but different |
| | slope) for Cases 1 (distributed) and 2 (localized) |

We next characterized the MORPH analytical performance. To evaluate assay sensitivity, exosomes were serially diluted and quantified by gold-standard NTA, before being analyzed for CD63 expression on the MORPH platform.

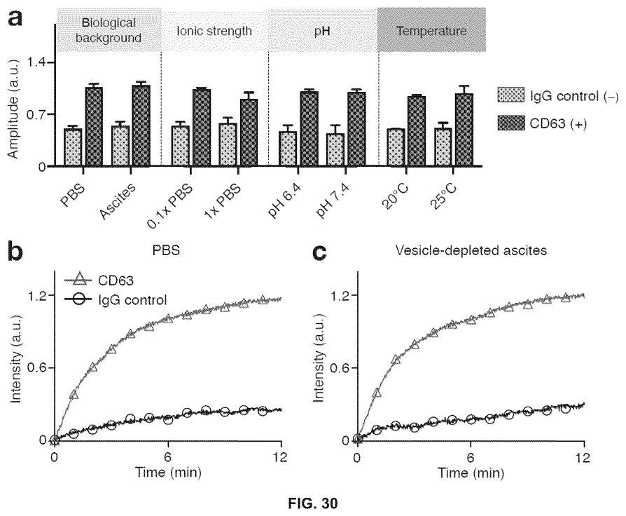

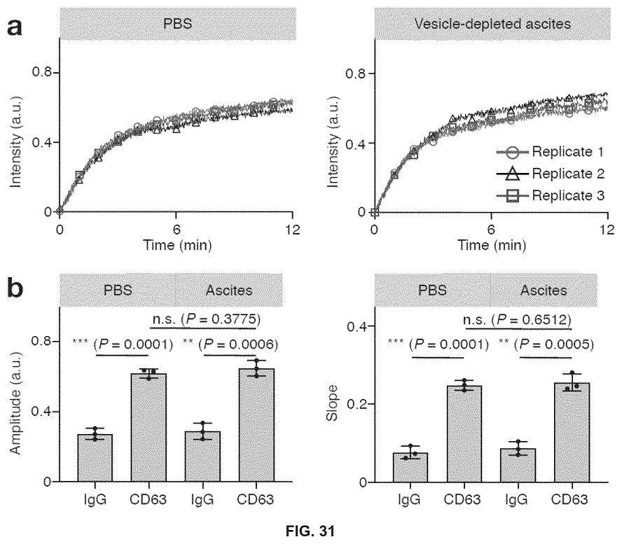

The critically-locked MORPH assay showed a LOD of ˜1700 exosomes, which is 10³-fold better than that of conventional ELISA (LOD: ˜1×10⁶ exosomes), and 10⁴-fold better than the unlocked metamaterial analysis (LOD: ˜2×10⁷ exosomes) (FIG. 26c). When assessing vesicles derived from different cell origins and for different target biomarkers (CD63, EpCAM, CD24 and MUC1), the MORPH analysis showed a good correlation (R²=0.947) to the gold-standard ELISA measurements (FIGS. 26d and 27c-d). Importantly, the MORPH assay showed biomarker-specific measurements, which were minimally influenced by different chemical agents and physical effects (biological background, buffer ionic strength, pH and temperature) (FIG. 30). When activated with a low concentration of exosomes, MORPH demonstrated rapid and reproducible signals (FIG. 31).

### Example 10. Clinical Utility of MORPH

To assess the clinical utility of MORPH, we finally conducted a feasibility study using cancer patient ascites. We aimed to determine (1) if the MORPH platform could be directly applied to clinical specimens for informative exosome analysis (e.g. biomarker abundance and distribution), and (2) the accuracy of MORPH-revealed signatures in distinguishing patient prognosis. We obtained cancer ascites samples (n=38) and used the miniaturized MORPH platform (FIGS. 3c-e) to perform exosome analysis directly in clinical samples (5 μL for each native sample). NTA was performed by following the protocol in Example 9.

### Clinical Measurements

The study was approved by the National University Hospital (2005/00440 and 2016/01088) and SingHealth (2015/2479) Institutional Review Boards. All subjects were recruited according to IRB-approved protocols after obtaining informed consent. Ascites samples were collected from colorectal cancer and gastric cancer patients, centrifuged at 500 g for 10 min, and filtered through a 0.2-μm membrane filter (Millipore). All samples were de-identified and stored at −80° C. before MORPH measurements.

For clinical MORPH analysis, ascites samples were used directly. We applied the ascites samples to the critically-locked metamaterial for MORPH analysis. For all MORPH measurements, we included an IgG isotype control antibody (as described in the MORPH workflow for exosome molecular profiling protocol in Example 9). MORPH analysis was performed relative to this control to account for nonspecific binding of antibodies. Clinical evaluation of patient characteristics was determined independently. Specifically, patient prognosis was determined by the overall survival from the time of collection of ascites. In our clinical cohort, patient survival ranged from <1 month to 53.3 months, with a median survival of 10.17 months (59.2% patient survived less than 10 months, 40.8% survived more than 10 months). This is consistent with published reports, where patient survival ranged from <1 month to 48 months (64.2% patient survived less than 10 months, 35.8% survived more than months) (Ayantunde, A. & Parsons, S., Ann. Oncol. 2007, 18, 945-949). Based on these data and published reports, we thus determined the cut-off for good prognosis as survival>months. All MORPH measurements were performed blinded from these clinical evaluations.

### Statistical Analysis for Clinical Correlation

For clinical correlation, we used patient survival data as prognosis classifiers (good prognosis and poor prognosis), and performed leave-one-out cross validation to develop a multiple regression scoring model for establishing the combined biomarker signature. Specifically, we used the clinical MORPH measurements (biomarker-associated amplitude and slope) to develop the multiple regression scoring model for the classification of disease prognosis:

$$Score ( n ) = {\sum }_{i = 1}^{4} {α}_{i} \cdot {A}_{i} + {\sum }_{i = 1}^{4} {β}_{i} \cdot {S}_{i} + {γ}_{0} \qquad ( 1 )$$

where Aᵢ and Sᵢ are the measured amplitude and slope for individual markers, αᵢ and βᵢ are their corresponding regression coefficients, and γ₀ is the y-intercept. Regression coefficients and their respective P-values are listed in Table 3. To evaluate the clinical correlation, we performed receiver operating characteristic (ROC) curve analysis of individual biomarkers as well as the combined signature, and computed the values of AUC using the trapezoidal rule. Using the Youden's index to define the optimal assay threshold, we assessed the MORPH assay's sensitivity, specificity and accuracy. Statistical analyses were performed using MATLAB (2018a) and GraphPad Prism (v.7.0c).

TABLE 3
List of regression coefficients.

| | α₁ | α₂ | α₃ | α₄ | β₁ | β₂ | β₃ | β₄ | γ₀ |
|---|---|---|---|---|---|---|---|---|---|
| | (CD63, | (CD24, | (EpCAM, | (MUC1, | (CD63, | (CD24 | (EpCAM, | (MUC1, | (y- |
| | amplitude) | amplitude) | amplitude) | amplitude) | slope) | slope) | slope) | slope) | intercept) |
| Regressioncoefficient | −0.889 | 0.951 | 0.532 | 0.480 | 0.272 | 0.585 | 0.390 | 0.177 | 0.282 |
| P-value | 0.001 | 0.028 | 0.044 | 0.289 | 0.038 | 0.048 | 0.049 | 0.181 | 3.1e⁻⁵ |

### Results and Discussion

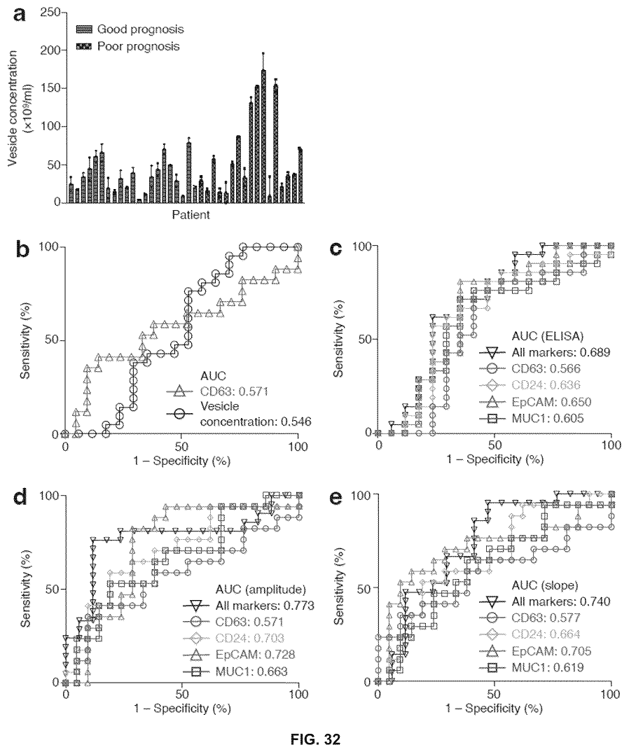

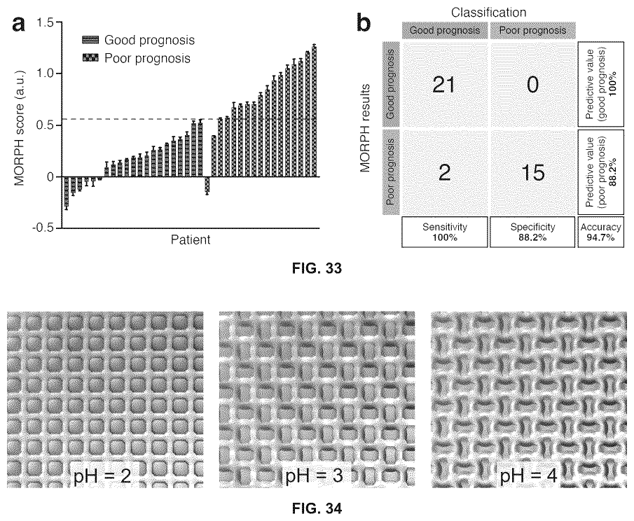

Specifically, we performed the MORPH analysis on three putative cancer markers (CD24, EpCAM and MUC1) as well as an exosome marker (CD63) (FIG. 26e). For each biomarker, we leveraged the MORPH's amplitude and slope analyses to characterize the clinical samples. Using patient survival data, as determined from the length of survival after ascites collection, we used the multiparametric MORPH measurements to develop regression scoring models for classifying patient prognosis and validated these models using leave-one-out cross-validation (FIG. 26f). Amongst all vesicle analyses performed (e.g., total vesicle counts) (FIG. 32), the MORPH's combined signature (FIG. 26g, AUC=0.941) demonstrated the best accuracy for prognosis classification, than that using individual biomarkers (amplitude or slope analysis of individual biomarkers) (AUC<0.773) or by ELISA analysis (AUC<0.689). We further evaluated the performance of the MORPH signature for clinical stratification (FIG. 33). Using the Youden's index to define the optimal assay threshold, the MORPH platform demonstrated 100% sensitivity (21/21), 88.2% specificity (15/17) and an accuracy of 94.7% (36/38) in differentiating patient prognosis.

Taken together, the MORPH platform is robust and sensitive. Different-state metamaterials can be precisely tuned and locked to their respective critical states, regardless of their initial preparation, to enhance and distinguish different hydrogel responses (swelling vs. cross-linking). The resultant MORPH signals are not only amplified in magnitude but are also fast in response, demonstrating rapid and localized kinetics. We thus developed the technology for molecular profiling of whole exosomes, a class of circulating extracellular vesicles with a typical diameter of 30-200 nm. Leveraging both amplitude and kinetic analyses, we applied the MORPH technology to characterize biomarker composition of these nanoscale vesicles. The developed system not only achieved sensitive quantification (10³-fold improvement over ELISA, 5 μL of sample in 15 min), but also distinguished vesicle mixtures with different biomarker distribution. When employed to examine native patient ascites, the technology revealed exosome molecular signatures against a complex biological background to accurately differentiate cancer patient prognosis.

### Example 11. pH Responsive Hydrogel

### Hydrogel Composition:

2-hydroxyethyl methacrylate (HEMA, base component for good mechanical property); and
acrylic acid monomer (AA, pH responsive component).

The incorporated acrylic acid component is pH responsive (less swollen at low pH). As shown in FIG. 34, the hydrogel structure was in an unbuckled state at pH=2, and then turned into a buckled state when the solution pH was increased to 3 and 4.

### Comparative Example 1

In comparison to conventional hydrogel biosensors (Table 4), the MORPH offers advantages with respect to both hydrogel optimization and sensing mechanism. Firstly, for hydrogel optimization, while conventional hydrogel biosensors rely on pre-casting optimization (e.g., tuning of hydrogel material composition and/or casting condition), MORPH uses post-casting modulation to tune both the hydrogel's molecular and geometric properties (e.g. tuning of the hydrogel's swelling after metamaterial casting); specifically, we incorporate a temperature-responsive component (NIPAM) into the hydrogel network, and apply plasmonic heating to tune the already-casted metamaterial to its most responsive critical state (i.e. by maximizing the cured hydrogel's molecular-level mechanical strain while preserving its geometric pattern). As the critical point in mechanical metamaterial is a highly delicate state, conventional pre-casting optimization faces significant challenges in attaining this state, likely due to variable cross-linking and/or patterning during hydrogel preparation (FIG. 8e). Our post-casting modulation is robust and precise; it tunes the casted metamaterial to its critical transition state, regardless of its initial casted states or storage conditions, to support high responsiveness that can be easily missed with conventional preparation. Drawing on this robustness and precision, the entire MORPH assay workflow can be readily implemented on a miniaturized microfluidic platform.

TABLE 4
Comparison of detection technologies.

| | | Unstructured | Structured | Plasmonic | Electrochemical | |
|---|---|---|---|---|---|---|
| | MORPH | hydrogel sensor | hydrogel sensor | sensor | sensor | ELISA |
| Sensing | Swelling-induced | Swelling-induced | Swelling-induced | Refractive | Redox reaction- | Chemiluminescence |
| mechanism | pattern | volumetric change | volumetric change | index change | induced current | |
| | transformation | (Cai, Z. et al., | (MacConaghy, K. I. | (Lim, C. Z. J. | (Park, J. et al., | |
| | | ACS Sens. 2017, 2, | et al., Anal. Chem. | et al., Nat. | ACS Nano 2017, | |
| | | 1474-1481; and | 2015, 87, 3467-3475; | Commun. 2019, | 11, 11041-11046; | |
| | | Miyata, T., Asami, | Zhao, J. et al., Ind. | 10, 1144; and | Yadav, S. et al., | |
| | | N. & Uragami, T., | Eng. Chem. Res. 2020, | Im, H. et al., | ChemElectro Chem | |
| | | Nature 1999, 399, | 59, 10469-10475; and | Nat. Biotechnol. | 2017, 4, 967-971; | |
| | | 766-769) | Zhang, J. T. et al., | 2014, 32, 490-495) | and Jeong, S. et | |
| | | | Chem. Commun. 2013, | | al., ACS Nano 2016, | |
| | | | 49, 6337-6339) | | 10, 1802-1809) | |
| Hydrogel | Mechanical | Bulk state | Regular structure, | — | — | — |
| geometry | metamaterial | | but no metamaterial | | | |
| | | | features | | | |
| Geometric | Rapid, nonlinear | Slow, linear | Slow, linear | — | — | — |
| deformation | transformation | | | | | |
| | at the critical | | | | | |
| | point | | | | | |
| Tuning of | Yes | Yes | Yes | — | — | — |
| hydrogel | | | | | | |
| property | | | | | | |
| (pre-casting) | | | | | | |
| Tuning of | Yes | No | No | — | — | — |
| hydrogel | (critical | | | | | |
| property | locking) | | | | | |
| (post-casting) | | | | | | |
| Implementation | Moderate | Minimal | Moderate | High | High | Moderate-High |
| requirement* | | | | | | |
| Sensitivity | High (~1700 | Low (0.1-1 μM | Low (~10-100 nM | High (~10²-10³ | Moderate (~10⁴- | Low (~10⁶ |
| (Limit of | vesicles) | protein or 10⁷- | protein or 10⁶-10⁷ | vesicles) | 10⁵ vesicles) | vesicles) |
| detection) | | 10⁸ vesicles\hat{ }) | vesicles\hat{ }) | | | |
| Ability to | Yes | No | No | Yes | No | No |
| measure vesicle | | | | | | |
| marker density | | | | | | |
| Assay | 15-30 min | ~2-24 h | ~2-24 h | ~1 h | ~1-2 h | >24 h |
| duration | | | | | | |

*Considers equipment requirements during device fabrication, sample preparation, and measurement.
\hat{ }Conversion is based on average protein amount of 10⁻¹³ g per extracellular vesicle (EV) (Patel, G. K. et al., Sci. Rep. 2019, 9, 5335).

Secondly, for sensing mechanism, while conventional hydrogel biosensors (e.g. the unlocked system) use stimulus-induced volumetric changes for detection, MORPH is enabled by its critical-locking to achieve stimulus-induced pattern transformation. Specifically, biological stimulus can readily perturb the critically-strained MORPH to trigger a rapid release of its accumulated strain energy; macroscopically, this induces a cooperative re-organization of the MORPH's geometric pattern to achieve an amplified diffraction signal. The resultant MORPH thus benefits from both versatile post-casting tuning (to attain the critical state) and amplified detection (stimulus-induced geometric transformation). This sensing mechanism also enables multi-selectivity of the system, leading to biomarker-specific chiral transformation. Firstly, for biomarker-selectivity, MORPH is extensively treated with blocking agents to reduce nonspecific binding; all measurements are also accompanied with sample-matched negative controls to measure biomarker-specific signals. Secondly, for transformation-selectivity, MORPH transforms only when the hydrogel metamaterial is tuned to its critical state and further reacts with peroxidase-generated free radicals. This process is selective as (1) plasmonic modulation in the casted metamaterial compensates for any variations in gel composition and/or environmental factors (e.g., temperature) to establish the system in a critical state, and (2) the generation and reaction of free radicals is highly specific and short-lived, to induce rapid and localized metamaterial cross-linking, thus making the system insensitive to other chemical variations (e.g. pH and salt concentration). Leveraging these attributes, we further developed the technology for informative exosome molecular profiling; by performing both end-point (amplitude) and real-time kinetic (slope) analyses, MORPH characterizes exosomes in native patient ascites, to reveal biomarker signatures for better patient stratification.

The technology has the potential to be expanded further. Through careful materials integration, especially from a rich repertoire of bio-responsive hydrogels, the technology could be readily advanced. For example, the incorporation of shape-changing DNA nanostructures within the metamaterial is likely to not only boost the responsiveness, but also provide new avenues to transduce and amplify even transient molecular interactions. Through structural design, beyond the current demonstration with a 2D-patterned metamaterial, the technology could be further developed by exploiting complex 3D architectures (e.g., auxetic, origami- or kirigami-inspired) and/or other types of metamaterials, thereby enabling the incorporation of more sophisticated transformation (e.g. topologically-polarized) and amplification (e.g. snap-through buckling and frustration-induced multistability) mechanisms to further enhance its analytical capability. With its hyper-responsive detection, MORPH could be applied to quantify low-abundance biomarkers, even from a small volume of clinical samples. Beyond biomarker abundance, we further anticipate that the technology could be expanded to evaluate different biomarker distribution. For example, protein interaction and aggregation could lead to different biomarker distribution states despite similar total abundance (monomeric vs. aggregated amyloid proteins in neurodegenerative diseases); the ability to distinguish such protein organizational states could empower novel biomarker discovery and improve our understanding of disease progression. With its demonstrated robustness in native patient specimens, MORPH could also be expanded to investigate diverse biomarkers, in various clinical biofluids (e.g., blood and urine) across a spectrum of diseases (e.g., infectious diseases, cancers and neurodegenerative diseases). Further technical improvements, through the incorporation of advanced microfluidics and arrayed sensor patternings, could facilitate highly-parallel biomarker measurements and large-scale clinical validation.

1. A composite material comprising:
a substrate; and
a patterned hydrogel disposed on the substrate, wherein:
the patterned hydrogel comprises stimulus-responsive constitutional units and constitutional units comprising one or more target molecule recognition moieties;
the stimulus-responsive constitutional units are responsive to a stimulus and are configured to produce a stress value S1 to the patterned hydrogel when the stimulus is applied;
the target molecule recognition moieties are responsive to a target molecule and are configured to impart a stress value S2 to the patterned hydrogel upon interaction with the target molecule;
the patterned hydrogel is configured to reversibly buckle and/or reversibly swell when a threshold stress level T of the patterned hydrogel is crossed.

2. The composite material according to claim 1, wherein the one or more target molecule recognition moieties comprise a moiety selected from the group consisting of a crosslinkable moiety, a cleavable crosslinking moiety, and a moiety capable of covalently bonding to a polar molecule.

3. (canceled)

4. (canceled)

5. The composite material according to claim 2, wherein the crosslinkable moiety is capable of being crosslinked by horseradish peroxidase.

6. The composite material according to claim 1, wherein the one or more target molecule recognition moieties comprise a moiety capable of reacting with a mixture of formaldehyde and tris(hydroxymethyl)aminomethane to form a functional group having the formula —CH2-NHC(CH2OH)3.

7. The composite material according to claim 1, wherein the patterned hydrogel comprises constitutional units comprising a moiety targeted to a biomolecule selected from the group consisting of a protein biomarker, a DNA sequence, and an RNA sequence.

8. The composite material according to claim 7, wherein the patterned hydrogel comprises constitutional units comprising a moiety targeted to a biomolecule that recruits an enzyme that is capable of catalysing a crosslinking reaction and/or a cleavage reaction.

9. The composite material according to claim 7, wherein the patterned hydrogel comprises constitutional units comprising a moiety targeted to a biomolecule selected from the group consisting of CD63, CD24, EpCAM, EGFR, MUC1, CD125, HER2 and CEA.

10. The composite material according to claim 1, wherein:
the one or more target molecule recognition moieties comprise a crosslinkable moiety and/or a cleavable crosslinking moiety; and
the patterned hydrogel comprises constitutional units comprising a bio-moiety that recruits an enzyme that is capable of catalysing a crosslinking reaction between the crosslinkable moieties, or a cleavage reaction of a cleavable crosslinking moiety, which crosslinking reaction or cleavage reaction imparts a stress value S2 to the patterned hydrogel.

11. The composite material according to claim 1, wherein:
(a) the constitutional units comprising one or more target molecule recognition moieties comprise constitutional units comprising a crosslinkable moiety selected from the group consisting of a phenol moiety and a thiol moiety,
or
(b) the constitutional units comprising one or more target molecule recognition moieties comprise constitutional units comprising a cleavable crosslinking moiety selected from the group consisting of a disulfide moiety and a thioketal moiety.

12. The composite material according to claim 1, wherein the composite material comprises a stimulus-transmitting layer disposed between the substrate and the patterned hydrogel, which stimulus-transmitting layer comprises a stimulus-transmitting material capable of transmitting a stimulus to the stimulus-responsive constitutional units,
where said stimulus-responsive constitutional units are responsive to said stimulus.

13. (canceled)

14. (canceled)

15. The composite material according to claim 1, wherein the stimulus-responsive constitutional units are selected from one or more of the group consisting of thermally-responsive constitutional units, electrically-responsive constitutional units, optically-responsive constitutional units, magnetic-responsive constitutional units and pH-responsive constitutional units.

16. (canceled)

17. The composite material according to claim 1, wherein the patterned hydrogel is patterned to have a lattice structure.

18. The composite material according to claim 1, wherein the reversible buckling and/or reversible swelling of the patterned hydrogel is detectable by scanning electron microscopy and/or laser diffraction.

19. A method of detecting a biomolecule target in a sample, comprising the steps:
(i) providing a composite material according to claim 1, and a source of a stimulus to which the stimulus-responsive material is responsive;
(ii) applying the stimulus at a first magnitude to the composite material in the presence of said sample;
(iii) repeating step (ii) at a different stimulus magnitude to determine the threshold stress level T;
(iv) contacting the composite material with a sample and simultaneously applying the stimulus at a magnitude for which:
$$S 1 < T and S 1 + S 2 > T , or$$
$$S 1 > T and S 1 + S 2 < T ;$$
(v) determining the presence of said biomolecule target upon detecting a conformational change of the patterned hydrogel, wherein
a change in buckling state indicates the presence of said biomolecule target.

20. The method according to claim 19, wherein the source of a stimulus to which the stimulus-responsive material is responsive is a source of thermal energy or a pH change.

21. The method according to claim 19, wherein the buckling and/or swelling of the patterned hydrogel is determined using scanning electron microscopy (SEM).

22. The method according to claim 19, wherein the conformational change of the patterned hydrogel is determined using laser diffraction.

23. The method according to any one of claim 19, wherein:
(a) the one or more target molecule recognition moieties comprise crosslinkable moieties; and
(b) the patterned hydrogel comprises constitutional units comprising a moiety targeted to a biomolecule that recruits an enzyme that is capable of catalysing a crosslinking reaction of the crosslinkable moieties.

24. The method according to claim 23, wherein the patterned hydrogel comprises constitutional units comprising a moiety targeted to a biomolecule selected from the group consisting of CD63, CD24, EpCAM, EGFR, MUC1, CD125.

25. The method according to claim 23, wherein the crosslinkable moieties comprise tyrosine moieties, and where the enzyme is horseradish peroxidase (HRP).
